(19) United States
(12) Patent Application Publication
(10) Pub. No.: US 2023/0416591 A1
(43) Pub. Date: Dec. 28, 2023

(54) DUAL CURE SYSTEM FOR THE CHEMICAL FASTENING OF AN ANCHORING MEANS IN A BOREHOLE

(71) Applicant: HILTI AKTIENGESELLSCHAFT, Schaan (LI)

(72) Inventors: Armin PFEIL, Landsberg am Lech (DE); Christian Slugovc, Graz (AT); Christina Wappl, Graz (AT)

(73) Assignee: HILTI AKTIENGESELLSCHAFT, Schaan (LI)

(21) Appl. No.: 18/252,366

(22) PCT Filed: Oct. 28, 2021
(86) PCT No.: PCT/EP2021/079999

§ 371 Date: May 10, 2023

(30) Foreign Application Priority Data
Nov. 10, 2020 (EP) 20206588.4

(51) Int. Cl. C09K 8/44; C08G 61/08; C08K 9/00; C07C 69/74; C07C 69/54; F16B 13/14; E21B 23/01

(52) U.S. Cl. CPC C09K 8/44; C08G 61/08; C08K 9/00; C07C 69/74; C07C 69/54; C07C 2602/42; E21B 23/01; C08G 2261/12; C08G 2261/3324; C08G 2261/1426; C08G 2261/418; F16B 13/142

(57) ABSTRACT

A two-component system can be used for the chemical fastening of an anchor in a borehole by dual cure curing. The dual cure curing used is a combination of radical polymerization and ring-opening olefin metathesis polymerization (ROMP).

The present invention relates to a two-component system for the chemical fastening of an anchoring means in a borehole by means of dual cure curing. The dual cure curing used is a combination of radical polymerization and ring-opening metathesis polymerization (ROMP).

### BACKGROUND

The use of chemical fastening agents based on radically curable polymers has long been known. In the field of fastening technology, the use of polymers as an organic binder for the chemical fastening technology, e.g. as a constituent of an anchor mass (“chemical anchor”), has prevailed. Anchor masses of this kind are composite masses which are packaged as multi-component systems, usually two-component systems (“2C systems”), with one component containing the radically curable monomer and the other component containing an initiator (for radical formation). Other common constituents such as additives, fillers, accelerators, inhibitors, solvents, and reactive diluents can be contained in one and/or the other component. By mixing the two components, the curing reaction. i.e. the polymerization, is initiated by radical formation and the mixture is cured to obtain duromers.

Either epoxy-amine systems or methacrylate-based systems are usually used as chemical anchors. The former have low polymerization shrinkage, but cure very slowly at low ambient temperatures, such as those found on construction sites, and often with insufficient degrees of curing. In addition, they have a high viscosity at low temperatures, which makes them difficult to use on construction sites. Furthermore, they are critical with regard to user safety and the environment, as they contain corrosive polyamine hardeners, and epoxy resins can be sensitizing and hazardous to water. That is why methacrylate-based systems are usually used on construction sites. Peroxides such as diacetyl peroxide, hydroperoxides or peroxyesters are typically used as initiators for the radical curing of methacrylate-based chemical anchors. The weak point of methacrylate-based chemical anchors is the significant shrinkage during polymerization, which can lead to shear stress and weakening of the chemical anchor. Polymerization shrinkage due to the denser packing of the atoms in the polymer compared to the monomers weakens the bond strength of the chemical anchor either because the polymer detaches from the borehole wall as a result of the shrinkage, and/or because the polymer remains attached to both the borehole wall and the anchor rod to be fastened despite the shrinkage, thus creating stress due to the shrinkage.

The object of the invention is to provide a methacrylate-based two-component system to be used for the chemical fastening of an anchoring means in a borehole, which system has low shrinkage.

This object is achieved by the subject matter of the claims.

### DESCRIPTION OF THE INVENTION

The invention relates to a chemical anchor system which combines ring-opening metathesis polymerization (ROMP) of a compound with a strained cycloolefin group, preferably with a norbornene group, with radical polymerization of a compound with a (meth)acrylate group (preferably a methacrylate group), for example of methyl methacrylate or butane-1,4-diol-dimethylacrylate (BDDMA).

This new approach combines the advantage of rapid curing and excellent reactivity, even at low curing temperatures of radical-initiated (meth)acrylate-based systems, with the advantage of low shrinkage in systems polymerized by ROMP. This is because the combination of radical polymerization with ROMP leads to covalently crosslinked thermoset materials with a polymerization shrinkage which is reduced compared to radical polymerization of (meth)acrylate-containing monomers as the only monomer, for example methyl methacrylate.

The system according to the invention is therefore a dual cure system. The dual cure system according to the invention is based on the initiation of two different polymerization reactions triggered by mixing the components of the system: radical-initiated polymerization (RadP) and ring-opening metathesis polymerization (ROMP). The dual cure system according to the invention is therefore also referred to below as a RadP-ROMP dual cure system.

The ROMP polymerization requires a ROMP monomer, i.e. a monomer comprising a strained cycloolefin as a structural element, and a ROMP initiator; and the RadP polymerization requires a (meth)acrylate-containing monomer (preferably a methacrylate-containing monomer) that can be radically polymerized and a radical initiator, which is typically combined with a radical accelerator.

The RadP-ROMP dual cure system according to the invention therefore requires the use of two different monomers:
a) a monomer comprising a strained cycloolefin as a structural element, for example a norbornene; and
b) a (meth)acrylate-containing monomer (preferably a methacrylate-containing monomer), for example methyl methacrylate.

In principle, monomers (a) and (b) are molecules that are separate from one another. In one embodiment, however, the strained cycloolefin group and the (meth)acrylate group can be present as structural elements in a single molecule.

The RadP-ROMP dual cure system according to the invention also requires the use of two different initiators:
a) a ROMP initiator such as a Grubbs catalyst; and
b) a radical initiator, typically in combination with an accelerator, for example dibenzoyl peroxide (BPO) in combination with N,N-dimethyl-para-toluidine (DMpT).

In the RadP-ROMP dual cure system, these two initiators should be present separately from the matching monomers, so that polymerization starts only when the system components are mixed. Furthermore, the radical initiator should be present separately from the radical accelerator so that polymerization starts only when the system components are mixed.

A first object of the invention is a two-component system comprising:
a component A comprising a monomer which contains a strained cycloolefin as a structural element (ROMP monomer), and a radical initiator (for example BPO), and
a component B comprising a (meth)acrylate, preferably a methacrylate (for example methyl methacrylate or BDDMA), an initiator for ring-opening metathesis polymerization (ROMP initiator), and a radical accelerator (for example DMpT).

Optionally, the two-component system according to the invention having components A and B can be part of a multi-component system which also includes further components. In this case, constituents of components A and B can also be distributed over one or more of the further components.

The two-component system according to the invention forms a polymer when the two components A and B are mixed. It is preferably used for the chemical fastening of an anchoring means in a borehole and is therefore a chemical anchor in a preferred embodiment.

A second object of the invention is a method for the chemical fastening of an anchoring means in a borehole, characterized in that a two-component system according to the invention is used for the fastening.

A third object of the invention is the use of a two-component system according to the invention for the chemical fastening of an anchoring means in a borehole, i.e. as a chemical anchor or part of a chemical anchor.

For better understanding of the invention, the following explanations of the terminology used herein are considered to be useful.

The abbreviations used mean (further abbreviations may be explained directly in the text): BDDMA: butane-1,4-diol dimethylacrylate; BHT: 2,6-di-tert-butyl-4-methylphenol (butylated hydroxytoluene); BPO: dibenzoyl peroxide; Cp: cyclopentadiene; Cy: cyclohexane or cyclohexyl; DCM: dichloromethane; DiPpT: N,N-diisopropyl-para-toluidine; DMpT: N,N-dimethyl-para-toluidine; eq: equivalents; EtOAc: ethyl acetate; HEMA: 2-hydroxyethyl methacrylate; M:I: monomer-initiator ratio; MES: mesityl. i.e. 2,4,6-trimethylphenyl; MMA: methyl methacrylate; SIMes: 1,3-bis (2,4,6-trimethylphenyl)-2-imidazolidinylidene; Tempol: 4-hydroxy-2,2,6,6-tetramethylpiperidinyloxyl; THF: tetrahydrofuran; TLC: thin-layer chromatography; vol. %: volume percent; v/v: volume fraction; wt. %: weight percent; w/w: weight proportion.

Within the meaning of the invention:
“ring-opening metathesis polymerization” or “ROMP” or “ROMP polymerization” or “ring-opening olefin metethesis polymerization” means a polymerization reaction based on olefin metathesis starting from a strained cycloolefin structure. A ROMP initiator, typically a metal-carbene complex, is used as the initiator of the polymerization. The strained cycloolefin contains at least one double bond in the ring. The double bond in the ring is redistributed with the aid of a metallacyclobutane intermediate, then ring opening and polymerization take place. The driving force behind the polymerization is the dissolution of the ring strain. See the following diagram:

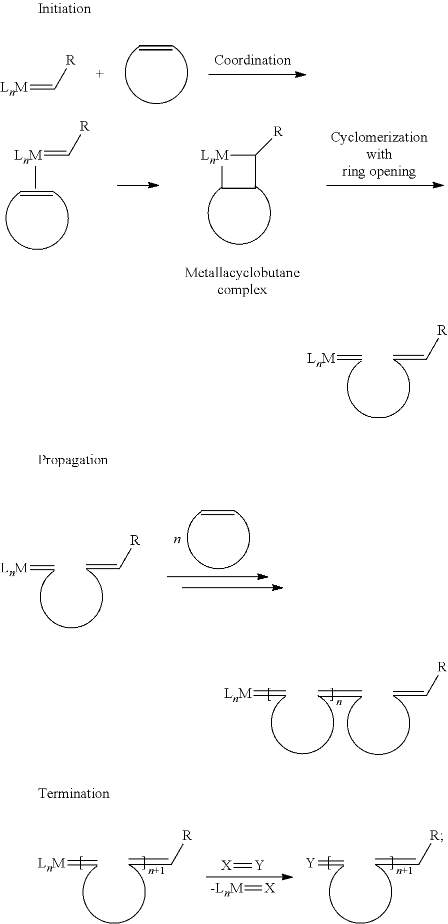

“RadP” or “RadP polymerization” means radically initiated polymerization. Radically initiated polymerization requires the formation of a radical in order to start the polymerization. Typical RadP polymerization is polymerization which is started by radical formation from BPO as a radical initiator in the presence of a tertiary amine such as DMpT as a radical accelerator:
“initiator” means a reagent that initiates polymerization;
“radical initiator” or “RadP initiator” means a reagent which (usually in combination with a radical accelerator) forms radicals that initiate radical polymerization;
“ROMP initiator” means a reagent which initiates and catalyzes ring-opening metathesis polymerization (ROMP), i.e. a catalyst for ring-opening olefin metathesis polymerization; the term “ROMP catalyst” is used synonymously here;
“accelerator” or “radical accelerator” means a reagent which reacts with a radical initiator so that larger amounts of radicals are generated by the radical initiator even at low temperatures, or which catalyzes the decomposition reaction of the radical initiator;
“inhibitor” means a substance which suppresses unwanted radical polymerization during the synthesis or storage of a monomer-containing composition (these substances are also referred to in the art as “stabilizers”) or which delays radical polymerization after the addition of a radical initiator, usually in conjunction with a radical accelerator (these substances are also referred to in the art as “inhibitors”—the particular meaning of the term becomes clear from the context);
“reactive diluents” means liquid or low-viscosity monomers which dilute other monomers and thereby impart the viscosity necessary for application thereof, which contain functional groups capable of reacting, and which for the most part become a constituent of the cured composition (e.g. of the mortar) in the polymerization (curing); reactive diluents are also referred to as co-polymerizable monomers;
“two-component system” or “2C system” means a system for producing a polymer that comprises two separately stored components, a component A and a component B, so that the monomers contained in the components cure only after the two components have been mixed;
“multi-component system” means a system for producing a polymer that comprises a plurality of components stored separately from one another, so that the monomers contained in the components cure only after all the components have been mixed;
“methacrylate” means an organic compound which contains a methacrylic acid ester group (for example methyl methacrylate, i.e. methacrylic acid methyl ester);
“(meth)acryl . . . / . . . (meth)acryl . . . ” means both the “methacryl . . . / . . . methacryl . . . ” and the “acryl . . . / . . . acryl . . . ” compounds; “methacryl . . . / . . . methacryl . . . ” compounds are preferred in the present invention;
“alkyl” means a saturated hydrocarbon group which can be branched or unbranched; preferably a C₁-C₂₀ alkyl, more preferably a C₁-C₉ alkyl. The group of C₁-C₂ alkyls includes, inter alia, methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, n-pentyl, iso-pentyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, 2-ethythexyl, n-octyl, iso-decyl, 2-propyl-heptyl, cyclohexyl, iso-nonyl, iso-tridecyl, 3,5,5-trimethyl-1-hexyl and 2-isopropyl-5-methylhexyl. Particularly preferred is a C₁-C₄ alkyl, i.e. an alkyl selected from the group consisting of methyl, ethyl, n-propyl, iso-propyl, n-butyl, iso-butyl, and tert-butyl; methyl, ethyl and tert-butyl are particularly preferred, and methyl is very particularly preferred;
“substituent,” unless stated otherwise, means a substituent that is not a hydrocarbon group, in particular a substituent selected from the group consisting of halogenyl, —OH, ═O, —CF₃, —CF₂—CF₃, —SiAlkyl₃, preferably from the group consisting of —F, —Cl, —OH, ═O and —CF₃; “substituted,” unless stated otherwise, means that one substituent or more substituents are present, preferably only one substituent;
“hydroxyalkyl” means an alkyl carrying at least one, preferably one or two hydroxyl groups, more preferably only one hydroxyl group, as a substituent, preferably on a carbon atom without further substituents;
“alkenyl” means an unsaturated hydrocarbon group having at least one, preferably only one, double bond, which can be branched or unbranched (but not cyclic); preferably a C₂-C₂₀ alkenyl, particularly preferably a C₂-C₆ alkenyl; i.e. an alkenyl selected from the group consisting of ethenyl, propenyl, butenyl, pentenyl and hexenyl; ethenyl, propenyl and butenyl are particularly preferred, and ethenyl is very particularly preferred;
“hydroxyalkenyl” means an alkenyl carrying at least one, preferably one or two hydroxyl groups as substituents, preferably on a carbon atom without further substituents and without a double bond to another carbon;
“alkynyl” means an unsaturated hydrocarbon group having at least one, preferably only one, triple bond, which can be branched or unbranched; preferably a C₂-C₂ alkynyl, particularly preferably a C₂-C₆ alkynyl, i.e. an alkynyl selected from the group consisting of ethynyl, propynyl, butynyl, pentynyl and hexynyl; ethynyl, propynyl and butynyl are particularly preferred, and ethenyl is very particularly preferred;
“hydroxyalkynyl” means an alkynyl carrying at least one, preferably one or two hydroxyl groups as substituents, preferably on a carbon atom without further substituents and without a triple bond to another carbon;
“aryl” or “Ar” means a mono- or bicyclic aromatic group, preferably with 6 to 10 carbon atoms. Exemplary C₆-C₁₀ aryl groups include phenyl and naphthyl. The aryl is preferably phenyl. The aryl group can optionally be substituted with one or more substituents selected independently of one another from the group consisting of —F, —Cl, —OH and —CF₃, —C₁-C₆ alkyl or —O—C₁-C₆ alkyl;
“heteroaryl” means a mono- or bicyclic aromatic group, preferably with 5 to 10 ring atoms, more preferably with 5 or 6 ring atoms, which contains at least one, preferably only one or two, particularly preferably only one heteroatom selected from S, O, N. and preferably N. Exemplary heteroaryl groups include pyrrole, pyridinyl, and pyrimidinyl. The heteroaryl group can optionally be substituted with one or more substituents selected independently of one another from the group consisting of —F, —Cl, —OH and —CF₃, —C₁-C₆ alkyl or —O—C₁-C₆ alkyl;
“arylalkyl” means a group of at least one aryl as defined above here with at least one alkyl as defined above here, which are linked together by a single bond. An arylalkyl is preferably an -(alkyl)ₙ-aryl-(alkyl)ₙ where n=0 or 1, particularly preferably it is —(CH₂)ₙ-phenyl-(CH₂)ₙ where n=0 or 1;
“a” or “an” as the article preceding a class of chemical compounds, e.g. preceding the word “methacrylate,” means that one or more compounds included in this class of chemical compounds, e.g. various methacrylates, may be intended. In a preferred embodiment, this article means only a single compound;
“at least one” means numerically “one or more.” In a preferred embodiment, the term numerically means “one”;
“contain,” “comprise,” and “include” mean that further constituents may be present in addition to those mentioned. These terms are intended to be inclusive and therefore also encompass “consist of.” “Consist of” is intended to be exclusive and means that no further constituents may be present. In a preferred embodiment, the terms “contain,” “comprise” and “include” mean the term “consist of”;
“approximately” or “approx.” before a numerical value means a range of ±10% of this value, preferably ±5% of this value, more preferably ±2% of this value, more preferably ±1% of this value, particularly preferably ±0% of this value (i.e. exactly this value);
a range limited by numbers, e.g. “from 10% to 20%,” means that the two extreme values and any value within this range are disclosed individually.

If a compound described herein has more than one stereoisomer in the form of tautomers, diastereomers, enantiomers. E/Z isomers, for example due to an asymmetric center or due to the possibility of an endo or exo configuration, then all possible stereoisomers and also mixtures are meant unless otherwise stated. For example, a di-substituted norbornene can be present in the endo/endo, endo/exo, or exo/exo configuration. In the case of compounds in which an endo/endo, endo/exo, or exo/exo configuration is possible, the endo/exo configuration is preferred. In the case of compounds in which an E or Z configuration is possible, in particular in the case of non-cyclic olefins which are to be used as one of the starting materials for the production of a strained cycloolefin by means of the Diels-Alder reaction (in particular with a cycloalk-1,3-diene), the E configuration is preferred.

If standards (e.g. DIN standards) are mentioned in this text, this refers to the current edition of the relevant standard on the filing date of this application.

The constituents of components A and B of the 2C system according to the invention are explained in more detail below.

Component A Component A comprises at least one monomer which contains a strained cycloolefin as a structural element (ROMP monomer), and at least one radical initiator (for example BPO).

ROMP Monomer

Component A comprises at least one ROMP monomer.

Monomers which are suitable for ROMP are generally known to a person skilled in the art, for example from Leitgeb A.; Wappel J.; Slugovc C., The ROMP toolbox upgraded, Polymer 2010, 51, 2927-2946; from Slugovc C., The Ring Opening Metathesis Polymerization Toolbox, Macromol. Rapid Commun. 2004, 25, 1283-1297; from EP 0 771 830 A2, and from U.S. Pat. No. 7,691,919 B2, columns 11 to 13. The description of the ROMP monomers given in these documents is hereby incorporated into the present description by reference.

A ROMP monomer can in principle be any compound which contains, as a structural element, at least one strained cycloolefin with at least one double bond in the ring, preferably only one double bond in the ring (“strained cycloolefin structural element”), or which is a strained cycloolefin. In addition to this at least one double bond, there are preferably no further multiple bonds (i.e. in particular no triple bonds) in the ring of the strained cycloolefin.

In the context of the present invention, “strained cycloolefin” refers to all cycloolefins with the exception of cyclohexene. Cyclohexene cannot be polymerized with ring-opening metathesis. Strained cycloolefins which are suitable for the present invention are disclosed, for example, in EP 0 771 830 A2 and in U.S. Pat. No. 7,691,919 B2, columns 11 to 13.

The strained cycloolefin (structural element) has at least one double bond in the ring, preferably one or two double bonds, more preferably only one double bond. If the strained cycloolefin (structural element) comprises two double bonds, these do not have a common carbon atom and are preferably not conjugated, and are more preferably part of different rings, or in the case of bridged systems part of different bridges, within the strained cycloolefin (structural element).

The strained cycloolefin (structural element) can contain at least one heteroatom, preferably only one heteroatom, selected from N, O, Si, P and S, preferably from N, O and S, more preferably O, as a ring atom. Preferably, however, all ring atoms are carbon atoms or only one of the ring atoms is replaced by O (oxa derivative), even more preferably all ring atoms are carbon atoms. In the case of an oxa derivative, —O— replaces a —CH₂—, in the case of a sulfa derivative —S— replaces a —CH₂—, N, Si and P can be linked to two or more carbon ring atoms, and the remaining valences are then linked to H atoms or substituents (e.g., —NH— or —N(CH₃)—).

The strained cycloolefin (structural element) is either monocyclic or polycyclic with two or more, preferably two to four, condensed, fused and/or bridged rings. It preferably comprises 3 to 16 ring atoms, more preferably 3 to 14 ring atoms, even more preferably 5 to 12, even more preferably 5, 7 or 8 ring atoms, even more preferably 5 or 7 ring atoms. If the strained cycloolefin (structural element) is monocyclic, it preferably comprises 3 to 8 ring atoms, more preferably 5 or 7 ring atoms; if it is polycyclic, in particular bicyclic, it preferably comprises 7 to 14 ring atoms, more preferably 7 to 12 ring atoms, even more preferably 7 to 9 ring atoms. In the case of polycyclic strained cycloolefins (structural element), bicyclo[x,y,z] olefins, tricyclo[w,x,y,z] olefins and tetracyclo[v,w,x,y,z] olefins are preferred. In which v, w, x, y and z are, independently of one another, an integer from 0 to 6, preferably an integer from 1 to 3, more preferably an integer from 1 to 2. Of these, bicyclo[x,y,z] olefins are particularly preferred, in particular for x=2, y=2, z=1 or 2.

The strained cycloolefin (structural element) is preferably monocyclic or bicyclic, preferably a monocyclic (for example cyclopentene) or bridged bicyclic strained cycloolefin (for example norbornene). A bicyclic strained cycloolefin is preferably a bicyclic strained cycloolefin which is formed by the Diels-Alder reaction of a monocyclic 1,3-diene with an olefin. If this olefin is di-substituted, it preferably has the E configuration, and the resulting strained cycloolefin has the endo/exo configuration, as shown in the following exemplary reaction:

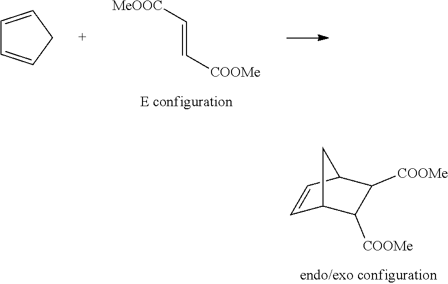 [SMILES: C1=CCC=C1 ; COC(=O)/C=C/C(=O)OC ; COC(=O)C1C2C=CC(C2)C1C(=O)OC]

Further strained cycloolefins formed by the Diels-Alder reaction of a cycloolefin with two double bonds in the ring (for example cyclopentadiene) with a cycloolefin (for example the tetracyclododecene formed from cyclopentadiene and norbornene, or dicyclopentadiene) also fall into the group of preferred strained cycloolefin structural elements. This includes in particular:

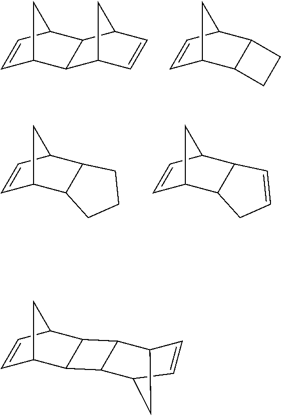

and their oxa derivatives.

In one embodiment, the strained cycloolefin (structural element) is selected from the group consisting of tricyclo[5.2.1.0^{2,6}]deca-3,8-diene (dicyclopentadiene), tetracyclo[6.2.1.1.0]-dodecene, bicyclo[3.3.2]-decene, bicyclo[3.2.2]-nonene, bicyclo[2.2.2]-octene, bicyclo[2.2.1]-heptadiene (norbornadiene), bicyclo[2.2.1]-heptene (norbornene) and bicyclo[2.2.0]-hexene, and their oxa derivatives.

The strained cycloolefin structural element is preferably a cyclopentene, a norbornene, a norbornadiene, a cyclooctene, a tricyclo[5.2.1.0^{2,6}]deca-3,8-diene (dicyclopentadiene) or a tetracyclo[6.2.1.1.0]-dodecene, or an oxa derivative thereof, more preferably a norbornene, a norbornadiene, a tricyclo[5.2.1.0^{2,6}]deca-3,8-diene (dicyclopentadiene), or a tetracyclo[6.2.1.1.0]-dodecene, or an oxa derivative thereof, more preferably a norbornene or norbornadiene, or an oxa derivative thereof, even more preferably a norbornene.

The ROMP monomer either consists of the unsubstituted, strained cycloolefin structural element. i.e. a strained cycloolefin, or the strained cycloolefin structural element can carry one or more, preferably one or two, substituents. In addition, at least one aryl ring, preferably a benzene ring, can be fused to the strained cycloolefin structural element.

In a preferred embodiment, the ROMP monomer is the unsubstituted, strained cycloolefin structural element, i.e. an unsubstituted mono- or polycyclic strained cycloolefin without a fused aryl; the ROMP monomer is therefore e.g. cyclopentene or norbornene.

In a further preferred embodiment, the ROMP monomer is the substituted, strained cycloolefin structural element, i.e. a substituted, strained cycloolefin. “Substituted, strained cycloolefin” means a strained cycloolefin structural element which is substituted with one or more, preferably one, substituents selected from the group consisting of halogen, pseudohalogen, C₁-C₂₀ alkyl, hydroxy-C₁-C₂₀-alkyl, C₂-C₂₀ alkenyl, hydroxy-C₂-C₂₀-alkenyl, C₂-C₂₀ alkynyl, hydroxy-C₂-C₂₀-alkynyl, —O—C₁-C₂₀-alkyl, —O—C₂-C₂₀-alkenyl, —O—C₂-C₂₀-alkynyl, —O—C(═O)—C₁-C₂₀-alkyl, —O—C(═O)—C₂-C₂₀-alkenyl, —O—C(═O)—C₂-C₂₀-alkynyl, —C(═O)—O—C₁-C₂₀-alkyl, —C(═O)—O—C₂-C₂₀-alkenyl, —C(═O)—C₂-C₂₀-alkynyl, —C(═)—O—C₁-C₂₀-hydroxyalkyl, —C(═O)—O—C₂-C₂₀-hydroxyalkenyl, —C(═O)—O—C₂-C₂₀-hydroxyalkynyl, —O—C₁-C₂₀-alkanediyl-C(═O)—O—C₁-C₂₀-hydroxyalkyl, —O—C₂-C₂₀-alkanediyl-C(═O)—O—C₁-C₂₀-hydroxyalkenyl, —O—C₁-C₂₀-alkanediyl-C(═O)—O—C₂-C₂₀-hydroxyalkynyl, —O—C₁-C₂₀-alkenyldiyl-C(═O)—O—C₁-C₂₀-hydroxyalkyl, —O—C₁-C₂₀-alkenyldiyl-C(═O)—O—C₂-C₂₀-hydroxyalkenyl, —O—C₁-C₂₀-alkenyldiyl-C(═O)—O—C₂-C₂₀-hydroxyalkynyl, —O—C₁-C₂₀-alkanediyl-O—C(═O)—C₂-C₂₀-alkyl, —O—C₁-C₂₀-alkanediyl-O—C(═O)—C₂-C₂₀-alkenyl, —O—C₁-C₂₀-alkanediyl-O—C(═O)—C₁-C₂₀-alkynyl, heteroaryl, —O-heteroaryl, —O—C₁-C₂₀-alkanediyl-heteroaryl, —O—C(═O)-heteroaryl, —O—C₁-C₂₀-alkanediyl-O—C(═O)-heteroaryl, aryl, —O-aryl, —O—C₁-C₂₀-alkanediyl-aryl, —O—C(═O)-aryl, —O—C₁-C₂₀-alkanediyl-O—C(═O)-aryl or combinations thereof.

The substituent is preferably selected from the group consisting of halogen, C₁-C₂₀ alkyl, hydroxy-C₁-C₂₀-alkyl, C₂-C₂₀ alkenyl, hydroxy-C₂-C₂₀-alkenyl, —O—C₁-C₂₀-alkyl, —O—C₂-C₂₀-alkenyl, —O—C₂-C₂₀-alkynyl, —O—C(═O)—C₁-C₂₀-alkyl, —O—C(═O)—C₂-C₂₀-alkenyl, —O—C(═O)—C₂-C₂₀-alkynyl, —O—C₁-C₂₀-alkanediyl-O—C(═O)—C₁-C₂₀-alkyl, and —O—C₁-C₂₀-alkanediyl-O—C(═O)—C₂-C₂₀-alkenyl. The substituent is particularly preferably an —O—C(═O)—C₂-C₂₀-alkenyl or O—C₁-C₂₀-alkanediyl-O—C(═O)—C₂-C₂₀-alkenyl.

The substituent is preferably bonded to a C atom of the strained cycloolefin structural element which is not part of the C═C double bond in the ring and also (in the case of a bicyclic situation) is not a bridgehead carbon atom. In a further preferred embodiment, the substituent in the case of a polycyclic strained cycloolefin is located on a ring without a double bond in the ring, or in the case of bridged systems on a bridge other than that in which the double bond is located.

In a preferred embodiment, the substituent is a structural element which is an ester of a carboxylic acid, preferably an α,β-unsaturated carboxylic acid (the resulting monomer is described separately below as a “RadP-ROMP monomer”). In a preferred embodiment, the esterified group is an alkyl or an alkenyl, or a hydroxyalkyl or hydroxyalkenyl, i.e. a group which, in addition to the —O— in the ester group, also carries one or more, preferably one, free OH group. In a further preferred embodiment, the carboxylic acid group is esterified with the strained cycloolefin element, either directly or indirectly. Indirectly means that there are further atoms between the ester group and the strained cycloolefin element, for example a branched or unbranched —C₁-C₂₀ alkanediyl group, —C₁-C₂₀-alkanediyl-O group, —C(═O)—O-alkanediyl group, -alkanediyl-C(═O)—O-alkanediyl group, -alkanediyl-O—C(═O)-alkanediyl group, —O—C(═O)-alkanediyl group or —C₂-C₂₀-alkenyldiyl group.

In a preferred embodiment, the ROMP monomer is a compound of formula (I)

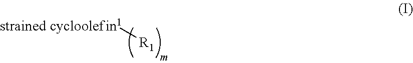

where:
strained cycloolefin means a strained cycloolefin structural element as defined above;
m is an Integer from 0 to 2, preferably 0 or 1; when m is 2, then the two R₁ are preferably the same;
R₁ are, independently of one another, selected from the group consisting of —C(═O)—OH, —C(═O)O—C₁-C₂₀-alkyl, —C(═O)—C₁-C₂₀-alkyl, —C(═O)—O-aryl, —O—C(═O)-aryl, —C₁-C₂₀-alkyl, -aryl, —C₁-C₂₀-hydroxyalkyl, —C₁-C₂₀-alkanediyl-O—C₁-C₂₀-alkyl, halogenyl, —CF₃, —CF₂—CF₃, and -SiAlkyl₃, R₁ is preferably a —C(═O)—OH, —C(═O)—O—C₁-C₈-alkyl, —O—C(═O)—C₁-C₈-alkyl, —C₁-C₈ alkyl, —C₁-C₈ hydroxyalkyl, or —C₁-C₈-alkanediyl-O—C₁-C₈-alkyl, more preferably a —C(═O)—OH, —C(═O)—O—C₁-C₄-alkyl, —O—C(═O)—C₁-C₄-alkyl, —C₁-C₄-Alkyl, —C₁-C₄-hydroxyalkyl, or —C₁-C₄-alkanediyl-O—C₁-C₄-alkyl, more preferably a —C(═O)—OH, —C(═O)—O—C₁-C₄-alkyl, —O—C(═O)—C₁-C₄-alkyl, —C₁-C₄ alkyl, —CH₂—OH, or —CH₂—O—C₁-C₄-alkyl. Even more preferably, R₁ is selected from the group consisting of —C(═O)—OH, —C(═O)—O-methyl, —O—C(═O)-methyl, methyl, ethyl, n-propyl, iso-propyl, n-butyl, iso-butyl, and tert-butyl, —CH₂—OH, —CH₂—O-methyl, 2-hydroxyethyl, 3-hydroxy-n-propyl, 2-hydroxy-isopropyl, 4-hydroxy-n-butyl, 3-hydroxy-isobutyl, and 2-hydroxy-tert-butyl. Methyl, ethyl, —C(═O)—O-methyl, —O—C(═O)-methyl, —CH₂—OH and 2-hydroxyethyl are particularly preferred, and methyl or —CH₂—OH is very particularly preferred.

Two R₁ bonded to adjacent carbon atoms can also be linked together and thus form a ring with the carbon atoms to which they are bonded, preferably a ring with 5 to 7 ring atoms. This can result in a cyclopentane ring or a lactone ring, for example.

Alternatively, two R₁ together form a bridge of formula —(CH₂)_{z}— which interconnects two cycloolefin structural elements, where z is an integer from 1 to 4, preferably from 1 to 3, more preferably from 1 to 2, particularly preferably 1, or a bridge of formula —(CH₂)_{q}—Ar—(CH₂)_{q}— which interconnects two cycloolefin structural elements, where Ar is an aromatic, preferably phenyl, q are, independently of one another, an integer from 0 to 2, preferably from 0 to 1, more preferably both are 0, or a bridge of formula —(CH₂)_{q}—C═C—(CH₂)_{q}— which interconnects two cycloolefin structural elements, where q are, independently of one another, an integer from 0 to 2, preferably from 0 to 1, more preferably both are 0, or a bridge of formula —(CH₂)_{q}—O—(CH₂), which interconnects two cycloolefin structural elements, where q are, independently of one another, an integer from 0 to 2, preferably from 0 to 1, even more preferably both are 0, or a bridge of formula —(CH₂)_{q}—NR₄—(CH₂)_{q}— which interconnects two cycloolefin structural elements, where q are, independently of one another, an integer from 0 to 2, preferably from 0 to 1, more preferably both are 0, and R₄ is H or a substituted or unsubstituted C₁-C₂₀ alkyl, more preferably H or an unsubstituted C₁-C₂₀ alkyl, even more preferably H, or a bridge of formula —C(═O)—CH₂)_{z}O—(O═)—C— which interconnects two cycloolefin structural elements, where z is an integer from 1 to 4, preferably from 1 to 3, more preferably from 1 to 2, particularly preferably 2, or a bridge of formula —C(═O)—O—, or a bridge of formula —C(═O)—O—Ar—O—(O═)—C— which interconnects two cycloolefin structural elements, where Ar is an aromatic, preferably phenyl, z is an integer from 1 to 4, preferably from 1 to 3, more preferably from 1 to 2, particularly preferably 1, or a bridge of formula —(CH₂)_{q}—Si(R₄)₂—(CH₂)_{q}— which interconnects two cycloolefin structural elements, where q are, independently of one another, an integer from 0 to 2, preferably from 0 to 1, more preferably at least once 1, and R₄ is H or a substituted or unsubstituted C₁-C₂₀ alkyl, more preferably H or an unsubstituted C₁-C₂₀ alkyl, more preferably an unsubstituted C₁-C₄ alkyl.

In a preferred embodiment (variant) the ROMP monomer has formula (I):

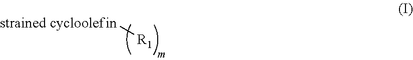

where:
strained cycloolefin is selected from the group consisting of cyclopentene, norbornene, norbornadiene, cyclooctene, tetracyclo[6.2.1.1.0]-dodecene and their oxa derivatives, in which a C atom in the strained cycloolefin structural element is replaced by O. It is preferably selected from the group consisting of norbornene, norbornadiene, tricyclo[5.2.1.0^{2,6}]deca-3,8-diene (dicyclopentadiene), tetracyclo[6.2.1.1.0]-dodecene, and their oxa derivatives, in which a C atom in the strained cycloolefin structural element is replaced by O;
m is 0 or 1;
R₁ is selected from the group consisting of —C(═O)—OH, —C(═O)—O—C₁-C₄-alkyl, —O—C(═O)—C₁-C₄-alkyl, —C₁-C₄alkyl, —CH₂—OH, or —CH₂—O—C₁-C₄-alkyl, and preferably is selected from the group consisting of —C(═O)—OH, —C(═O)—O-methyl, —O—C(═O)-methyl, methyl, ethyl, n-propyl, iso-propyl, n-butyl, iso-butyl, and tert-butyl, —CH₂—OH, —CH₂—O— methyl, 2-hydroxyethyl, 3-hydroxy-n-propyl, 2-hydroxy-Isopropyl, 4-hydroxy-n-butyl, 3-hydroxy-isobutyl, and 2-hydroxy-tert-butyl. Methyl, ethyl, —CH₂—OH and 2-hydroxyethyl are particularly preferred, and methyl is very particularly preferred. Alternatively, two R₁ together form a bridge of formula —(CH₂)_{z}— which interconnects two cycloolefin structural elements, where z is an integer from 1 to 2, preferably 1, or a bridge of formula —(CH₂)_{q}—C═C—(CH₂)_{q}— which interconnects two cycloolefin structural elements, where q are, independently of one another, an integer from 0 to 1, more preferably both are 0, or a bridge of formula —(CH₂)_{q}—O—(CH₂)_{q}—, which interconnects two cycloolefin structural elements, where q are, independently of one another, an integer from 0 to 2, preferably from 0 to 1, even more preferably both are 0, or a bridge of formula —C(═O)—O—(CH₂)_{z}—O—(O═)—C— which interconnects two cycloolefin structural elements, where z is an integer from 1 to 4, preferably from 1 to 3, more preferably from 1 to 2, particularly preferably 1, or a bridge of formula —C(═O)—O-phenyl-O—(O═)—C-which interconnects two cycloolefin structural elements, or a bridge of formula —(CH₂)_{q}—Si(R₄)₂—(CH₂)_{q}— which interconnects two cycloolefin structural elements, where q are, independently of one another, an integer from 0 to 1, more preferably at least once 1, and R₄ is H or a substituted or unsubstituted C₁-C₄ alkyl, more preferably H or an unsubstituted C₁-C₄ alkyl, more preferably an unsubstituted C₁-C₄ alkyl.

In a more preferred embodiment, the ROMP monomer has the following formula (II A) or (II B):

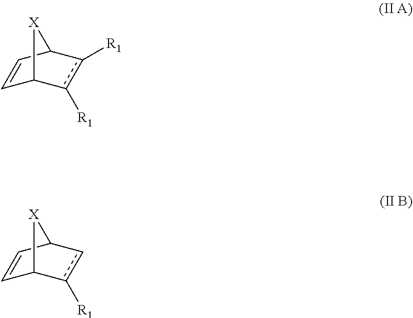

where:
[symbol] is a single or double bond, preferably a single bond;
R₁ is as defined above for formula (I) and preferably as defined for the preferred variant of formula (I) defined above, where the R1 in formula (II A) can be the same or different, but are preferably the same;
X is —CH₂— or —O—, preferably —CH₂—;
q is an integer selected from the group consisting of 1, 2, 3, 5 and 6, preferably selected from 1, 3 and 5, particularly preferably 3.

Very particularly preferred ROMP monomers are selected from the group consisting of

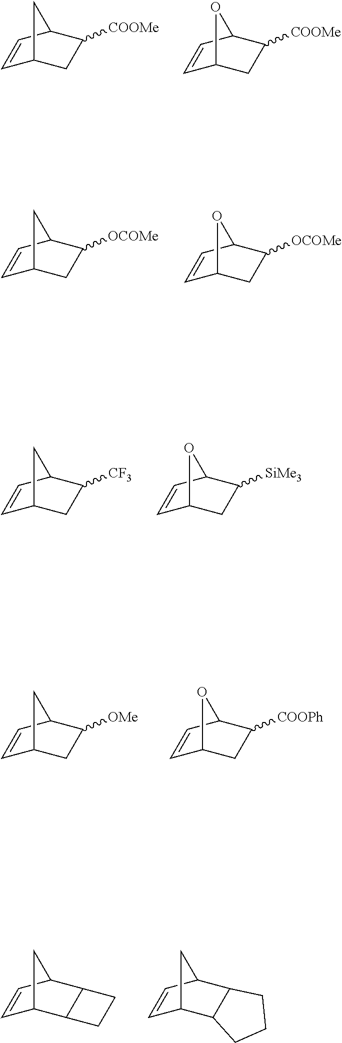

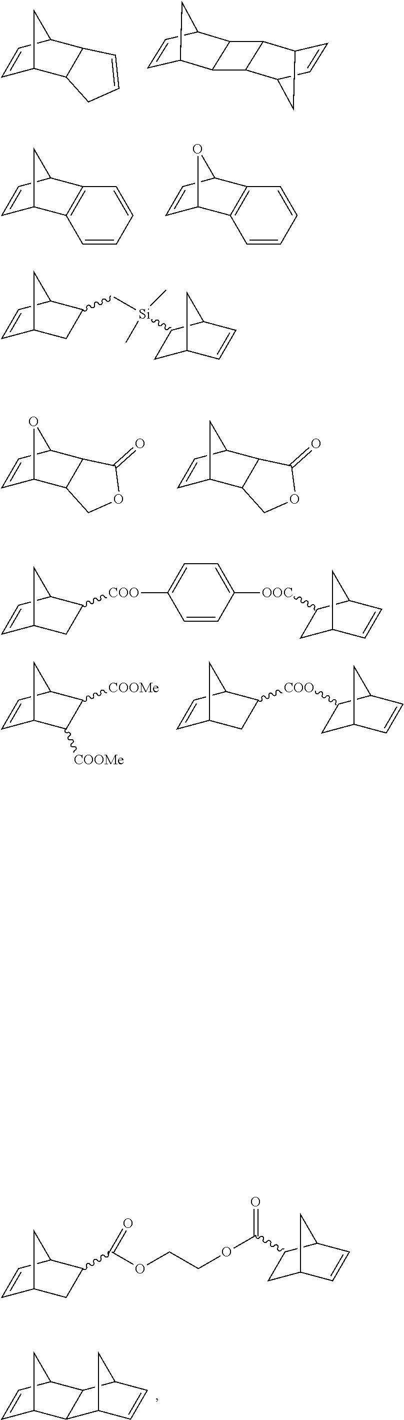

bicyclo[3.3.2]-decene, bicyclo[3.2.2]-nonene, bicyclo[2.2.2]-octene, bicyclo[2.2.1]-heptene (norbornene), bicyclo[2.2.0]-hexene, cyclopentene, cyclooctene, tetracyclo[8.2.1.1.0]-dodecene, tricyclo[5.2.1.0^{2,6}]deca-3,8-diene (dicyclopentadiene), and bicyclo[2.2.1]-heptadiene (norbornadiene) and their oxa derivatives, in which a C atom in the strained cycloolefin structural element is replaced by O. Further preferred from this group is the group consisting of

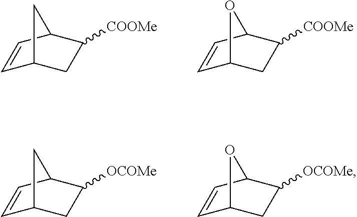

cyclopentene, norbornene, norbornadiene, cyclooctene, tetracyclo[6.2.1.1.0]-dodecene and their oxa derivatives, in which a C atom in the strained cycloolefin structural element is replaced by O. The group consisting of norbornene, norbornadiene, tricyclo[5.2.1.0^{2,6}]deca-3,8-diene (dicyclopentadiene), tetracyclo[6.2.1.1.0]-dodecene, and their oxa derivatives, in which a C atom in the strained cycloolefin structural element is replaced by O, is even more preferred. Most preferred is a norbornene or a ROMP monomer which comprises a norbornene structural element, or one of their oxa derivatives, in which a C atom in the strained cycloolefin structural element is replaced by O. Most particularly preferred is a norbornene or a ROMP monomer which comprises a norbornene structural element.

In one embodiment, the ROMP monomers from this very particularly preferred group are substituted with one of the substituents defined above. In another embodiment, they are not further substituted.

In a particular embodiment, the ROMP monomer is a RadP-ROMP monomer. A RadP-ROMP monomer is a ROMP monomer as defined above which, in addition to the strained cycloolefin structural unit, also contains a structural element which is an ester of an α,β-unsaturated carboxylic acid.

The α,β-unsaturated carboxylic acid, which is esterified in the RadP-ROMP monomer, is preferably selected from the group consisting of branched and unbranched C₃-C₂₀-α,β-unsaturated carboxylic acids, more preferably from the group consisting of branched and unbranched C₄-C₁₀-α,β-unsaturated carboxylic acids, more preferably from the group consisting of branched and unbranched C₄-C₂₀-α,β-unsaturated carboxylic acids. It is particularly preferably selected from the group consisting of tiglic acid ((E)-2,3-dimethylacrylic acid), sorbic acid (hexadienoic acid), crotonic acid (trans-butenoic acid), and methacrylic acid. More preferably it is methacrylic acid.

In this preferred embodiment, in which the ROMP monomer contains, in addition to the strained cycloolefin structural element, a structural element which is an ester of an α,β-unsaturated carboxylic acid, the two monomer elements required for the dual cure system, i.e. the strained cycloolefin monomer and the (meth)acrylate monomer, are already bonded to one another in a single monomer (hereinafter: RadP-ROMP monomer). Therefore, when using this (bifunctional) RadP-ROMP monomer, the presence of a further, separate monomer (for example in component B) is not absolutely necessary. In a preferred embodiment, however, in addition to the RadP-ROMP monomer, a (meth)acrylate is also present as a separate monomer in the 2C system according to the invention, typically in component B.

The use of a RadP-ROMP monomer allows two different types of polymerization with the same monomer molecule, namely ring-opening metathesis polymerization (ROMP) and radical polymerization (RadP). The production and the reaction possibilities of a RadP-ROMP monomer are shown in the following diagram as an example for the compound “Mon13.” 2-(methacryloyloxy)ethyl-endo,exo-5-norbornene carboxylate (“Mon13”) is produced in step B from 2 hydroxyethyl-endo,exo-5-norbornene carboxylate (“Mon9”), produced by esterification of the Diels-Alder product of 2-hydroxyethyl acrylate (HEA) and cyclopentadiene (Cp) (step A).

Then ROMP (step C. e.g. using a Grubbs catalyst) and radical polymerization (RadP) (step D, e.g. using benzoyl peroxide) are started simultaneously:

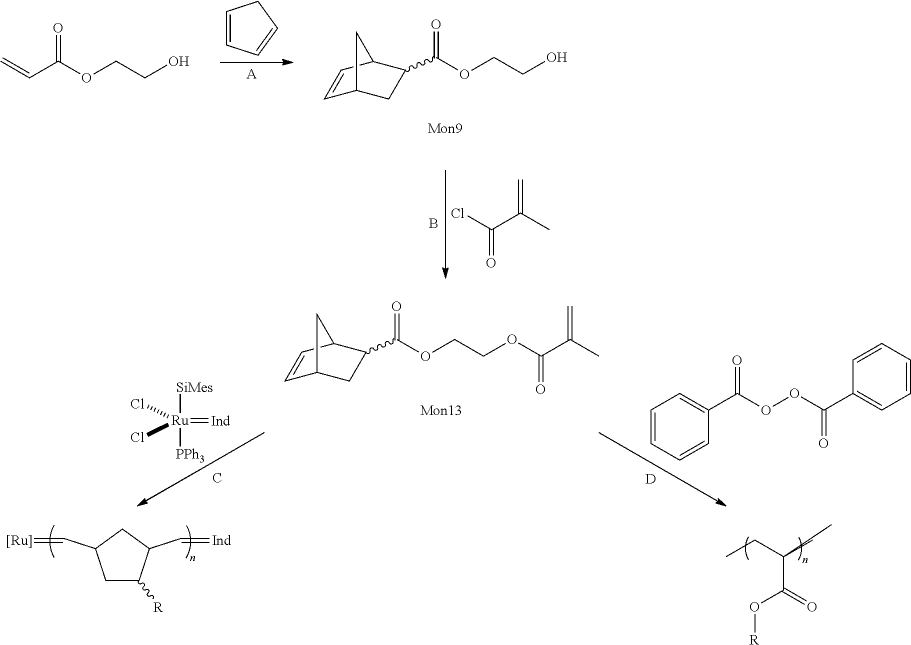

Crosslinking is achieved by the two different functional groups present in the molecule, as shown here for two RadP-ROMP monomers described in the examples, namely Mon13 and Mon14:

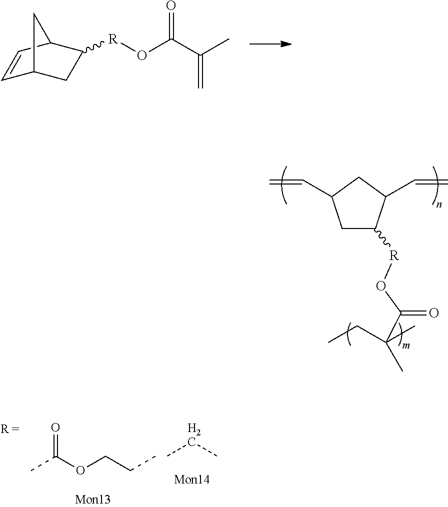

The α,β-unsaturated carboxylic acid is preferably selected from the group consisting of branched and unbranched C₃-C₂₀-α,β-unsaturated carboxylic acids, more preferably from the group consisting of branched and unbranched C₄-C₁₀-α,β-unsaturated carboxylic acids, more preferably from the group consisting of branched and unbranched C₄-C₆-α,β-unsaturated carboxylic acids. The α,β-unsaturated carboxylic acid is particularly preferably selected from the group consisting of acrylic acid, tiglic acid ((E)-2,3-dimethylacrylic acid), sorbic acid (hexadienoic acid), crotonic acid (trans-butenoic acid), and methacrylic acid. The α,β-unsaturated carboxylic acid is even more preferably methacrylic acid.

In a preferred embodiment, the RadP-ROMP monomer has the following formula (III):

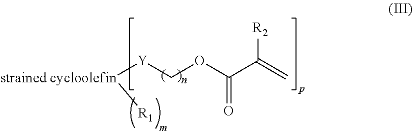

where:
strained cycloolefin means a strained cycloolefin structural element as defined above;
Y is selected from the group consisting of a single bond, —C₁-C₂₀-alkanediyl-, —O—C₁-C₂₀-alkanediyl-, —C₁-C₂₀-alkanediyl-O—, —O—C₁-C₂₀-alkanediyl-O—, —O—, —Si(alkyl)₂-, —C₁-C₂₀-alkanediyl-Si(alkyl)₂-, —C₁-C₂₀-alkanediyl-Si(alkyl)₂-C₁-C₂₀-alkanediyl-, —C₁-C₂₀-alkanediyl-O—C₁-C₂₀-alkanediyl-, —C(═O)—O—, —C₁-C₂₀-alkanediyl-C(═O)—O—, —C(═O)—O—C₁-C₂₀-alkanediyl-, —C(═O)—O—C₁-C₂₀-alkanediyl-O—C(═O)—, —C₁-C₂₀-alkanediyl-C(═O)—O—C₁-C₂₀-alkanediyl-, —O—C(═O)—, —O—C(═O)—O—, —C₁-C₂₀-alkanediyl-O—C(═O)—, —O—C(═O)—C₁-C₂₀-alkanediyl-, —O—C(═O)—C₁-C₂₀-alkanediyl-C(═O)—O—, -aryldiyl-, —O-aryldiyl-, —O-aryldiyl-O—, -aryldiyl-O-aryldiyl-, —C(═O)—O-aryldiyl-, —C(═O)—O—aryldiyl-O—C(═O)—, -aryldiyl-C(═O)—O-aryldiyl-, —O—C(═O)-aryldiyl-, —O—C(═O)-aryldiyl-C(═O)—O—, —C₆-C₂₀-arylalkanediyl-O—C₆-C₂₀-arylalkanediyl-, —O—C₆-C₂₀-arylalkanediyl-O—, —C₆-C₂₀-arylalkanediyl-O—C₆-C₂₀-arylalkanediyl-, —C(═O)—O—C₆-C₂₀-arylalkanediyl-, —C(═O)—O—C₆-C₂₀-arylalkanediyl-O—C(═O)—, —C₆-C₂₀-arylalkanediyl-C(═O)—O—C₆-C₂₀-arylalkanediyl-, —O—C(═O)—C₆-C₂₀-arylalkanediyl-, —O—C(═O)—C₆-C₂₀-arylalkanediyl-C(═O)—O—, —(CH₂)_{q}—C═C—(CH₂)_{q}—, where q are, independently of one another, an integer from 1 to 20, preferably from 1 to 10, even more preferably from 1 to 2, —(CH₂)_{q}—O—(CH₂)—, where q are, independently of one another, an integer from 1 to 20, preferably from 1 to 10, more preferably from 1 to, —(CH₂)_{q}—NR₄—(CH₂)_{q}—, where q are, independently of one another, an integer from 1 to 20, preferably from 1 to 10, even more preferably from 1 to, and R₄ is H or a substituted or unsubstituted C₁-C₂₀ alkyl, more preferably H or an unsubstituted C₁-C₂₀ alkyl, even more preferably H.
Y is preferably selected from the group consisting of a single bond, —C₁-C₈-alkanediyl-, —O—C₁-C₈-alkanediyl-, —C₁-C₈-alkanediyl-O—, —O—C₁-C₈-alkanediyl-O—, —O—, —SiMe₂-, —C₁-C₈-alkanediyl-SiMe₂, —C₁-C₈-alkanediyl-SiMe₂-C₁-C₈-alkanediyl-, —C₁-C₈-alkanediyl-O—C₁-C₈-alkanediyl-, —C(═O)—O—, —C₁-C₈-alkanediyl-C(═O)—O—, —C(═O)—O—C₁-C₈-alkanediyl-, —C(═O)—O—C₁-C₈-alkanediyl-O—C(═O)—, —C₁-C₈-alkanediyl-C(═O)—O—C₁-C₈-alkanediyl-, —O—C(═O)—, —O—C(═O)—O—, —C₁-C₈-alkanediyl-O—C(═O)—, —O—C(═O)—C₁-C₈-alkanediyl-, —O—C(═O)—C₁-C₈-alkanediyl-C(═O)—O—, —C₆-C₁₀-aryldiyl-, —O—C₆-C₁₀-aryldiyl-, —O—C₆-C₁₀-aryldiyl-O—, —C₆-C₁₀-aryldiyl-O—C₆-C₁₀-aryldiyl-, —C(═O)—O—C₆-C₁₀-aryldiyl-, —C(═O)—O—C₆-C₁₀-aryldiyl-O—C(═O)—, —C₆-C₁₀-aryldiyl-C(═O)—O—C₆-C₁₀-aryldiyl-, —O—C(═O)—C₆-C₁₀-aryldiyl-, —O—C(═O)—C₆-C₁₀-aryldiyl-C(═O)—O—, —C₇-C₁₂-arylalkanediyl-O—C₇-C₁₂-arylalkanediyl-, —O—C₇-C₁₂-arylalkanediyl-O—, —C₁-C₁₂-arylalkanediyl-O—C₇-C₁₂-arylalkanediyl-, —C(═O)—O—C₁-C₁₂-arylalkanediyl-, —C(═O)—O—C₇-C₁₂-arylalkanediyl-O—C(═O)—, —C₇-C₁₂-arylalkanediyl-C(═O)—O—C₇-C₁₂-arylalkanediyl-, —O—C(═O)—C₇-C₁₂-arylalkanediyl-, —O—C(═O)—C₇-C₁₂-arylalkanediyl-C(═O)—O—, —(CH₂)_{q}—C═C—(CH₂)—, where q are, independently of one another, an integer from 1 to 20, preferably from 1 to 10, even more preferably from 1 to 2, —(CH₂)_{q}—O—(CH₂)_{q}— where q are, independently of one another, an integer from 1 to 20, preferably from 1 to 10, more preferably from 1 to, —(CH₂)_{q}—NR₄—(CH₂)_{q}—, where q are, independently of one another, an integer from 1 to 20, preferably from 1 to 10, even more preferably from 1 to, and R₄ is H or a substituted or unsubstituted C₁-C₂₀ alkyl, more preferably H or an unsubstituted C₁-C₂₀ alkyl, even more preferably H.
More preferably, Y is selected from the group consisting of a single bond, —C₁-C₈-alkanediyl-, —O—C₁-C₈-alkanediyl-, —C₁-C₈-alkanediyl-O—, —O—C₁-C₈-alkanediyl-O—, —C₁-C₈-alkanediyl-O—C₁-C₈-alkanediyl-, —C(═O)—O—, —C₁-C₈-alkanediyl-C(═O)—O—, —C(═O)—O—C₁-C₈-alkanediyl-, —C(═O)—O—C₁-C₈-alkanediyl-O—C(═O)—, —C₁-C₈-alkanediyl-C(═O)—O—C₁-C₈-alkanediyl-, —O—C(═O)—, —C₁-C₈-alkanediyl-O—C(═O)—, —O—C(═O)C₁-C₈-alkanediyl-, —O—C(═O)—C₁-C₈-alkanediyl-C(═O)—O—, —C₆-C₁₀-aryldiyl-, —O—C₆-C₁₀-aryldiyl-.
Y is more preferably selected from the group consisting of —C₁-C₈-alkanediyl-, —C₁-C₈-alkanediyl-O—, —C(═O)—O—, —C₁-C₈-alkanediyl-C(═O)—O—, —C(═O)—O—C₁-C₈-alkanediyl-, —O—C(═O)—, —C₁-C₈-alkanediyl-O—C(═O)—.
Y is more preferably selected from the group consisting of —C(═O)—O—, —C₁-C₂-alkanediyl-, —C₁-C₂-alkanediyl-O—, —C(═O)—O—C₁-C₂-alkanediyl-, —C₁-C₂-alkanediyl-C(═O)—O—, —C₁-C₂-alkanediyl-O—C(═O)—, Y is particularly preferably —CH₂—, —CH₂—O—, —C(═O)—O—, —CH₂—C(═O)—O—, —CH₂—O—C(═O)—, or —C(═O)—O—CH₂. Y is very particularly preferably —CH₇—O—, —C(═O)—O—, —CH₂—C(═O)—O—, or —CH₂—O—C(═O);
m is an integer from 0 to 2, preferably 0 or 1; when m is 2, then the two R₁ are preferably the same;
n is an integer from 1 to 10, preferably from 2 to 6, more preferably 2;
p is an integer from 1 to 2, preferably 1;
R₁ are, independently of one another, selected from the group consisting of —C(═O)—OH, —C(═O)—O—C₁-C₂₀-alkyl, —O—C(═O)—C₁-C₂₀-alkyl, —C(═O)—O-aryl, —O—C(═O)-aryl, —C₁-C₂₀-alkyl, -aryl, —C₁-C₂₀-hydroxyalkyl, —C₁-C₂₀-alkanediyl-O—C₁-C₂₀-alkyl, halogenyl, —CF₃, —CF₂—CF₃, and -SiAlkyA₃. R₁ is preferably a —C(═O)—OH, —C(═O)—O—C₁-C₈-alkyl, —O—C(═O)C₁-C₈C-alkyl, —C₁-C₈ alkyl, —C₁-C₈ hydroxyalkyl, or —C₁-C₈-alkanediyl-O—C₁-C₈-alkyl, more preferably a —C(═O)—OH, —C(═O)—O—C₁-C₄-alkyl, —O—C(═O)—C₁-C₄-alkyl, —C₁-C₄-Alkyl, —C₁-C₄-hydroxyalkyl, or —C₁-C₄-alkanediyl-O—C₁-C₄-alkyl, more preferably a —C(═O)—OH, —C(═O)—O—C₁-C₄-alkyl, —O—C(═O)—C₁-C₄-alkyl, —C₁-C₄ alkyl, —CH₂—OH, or —CH₂—O—C₁-C₄-alkyl. Even more preferably, R₁ is selected from the group consisting of —C(═O)—OH, —C(═O)—O-methyl, —O—C(═O)-methyl, methyl, ethyl, n-propyl, iso-propyl, n-butyl, iso-butyl, and tert-butyl, —CH₂—OH, —CH₂—O-methyl, 2-hydroxyethyl, 3-hydroxy-n-propyl, 2-hydroxy-isopropyl, 4-hydroxy-n-butyl, 3-hydroxy-isobutyl, and 2-hydroxy-tert-butyl. Methyl, ethyl, —C(═O)—O-methyl, —O—C(═O)-methyl, —CH₂—OH and 2-hydroxyethyl are particularly preferred, and methyl or —CH₂—OH is very particularly preferred.
Two R₁ bonded to adjacent carbon atoms can also be linked together and thus form a ring with the carbon atoms to which they are bonded, preferably a ring with 5 to 7 ring atoms. This can result in a cyclopentane ring or a lactone ring, for example.
Alternatively, two R₁ together form a bridge of formula —(CH₂)_{z}— which interconnects two cycloolefin structural elements, where z is an integer from 1 to 4, preferably from 1 to 3, more preferably from 1 to 2, particularly preferably 1, or a bridge of formula —(CH₂)_{q}—Ar—(CH₂)_{q}— which interconnects two cycloolefin structural elements, where Ar is an aromatic, preferably phenyl, q are, independently of one another, an integer from 0 to 2, preferably from 0 to 1, more preferably both are 0, or a bridge of formula —(CH₂)_{q}—C═C—(CH₂)_{q}— which interconnects two cycloolefin structural elements, where q are, independently of one another, an integer from 0 to 2, preferably from 0 to 1, more preferably both are 0, or a bridge of formula —(CH₂)_{q}—O—(CH₂)_{q}— which interconnects two cycloolefin structural elements, where q are, independently of one another, an integer from 0 to 2, preferably from 0 to 1, even more preferably both are 0, or a bridge of formula —(CH₂)_{q}—NR₄—(CH₂)_{q}— which interconnects two cycloolefin structural elements, where q are, independently of one another, an integer from 0 to 2, preferably from 0 to 1, more preferably both are 0, and R₄ is H or a substituted or unsubstituted C₁-C₂₀ alkyl, more preferably H or an unsubstituted C₁-C₂₀ alkyl, even more preferably H, or a bridge of formula —C(═O)—O—(CH₂)—O—(O═)—C— which interconnects two cycloolefin structural elements, where z is an integer from 1 to 4, preferably from 1 to 3, more preferably from 1 to 2, particularly preferably 2, or a bridge of formula —C(═O)—O—, or a bridge of formula —C(═O)—O—Ar—O—(O═)—C— which interconnects two cycloolefin structural elements, where Ar is an aromatic, preferably phenyl, z is an integer from 1 to 4, preferably from 1 to 3, more preferably from 1 to 2, particularly preferably 1, or a bridge of formula —(CH₇)_{q}—Si(R₄)—(CH₂)_{q}— which interconnects two cycloolefin structural elements, where q are, independently of one another, an integer from 0 to 2, preferably from 0 to 1, more preferably at least once 1, and R₄ is H or a substituted or unsubstituted C₁-C₂₀ alkyl, more preferably H or an unsubstituted C₁-C₂₀ alkyl, more preferably an unsubstituted C₁-C₄ alkyl;
R₂ is a C₁-C₁₇ alkyl or C₁-C₁₇ hydroxyalkyl. R₂ is preferably a C₁-C₈ alkyl or C₂-C₈ hydroxyalkyl, even more preferably a C-CA alkyl or C₂-C₄ hydroxyalkyl. Even more preferably, R₂ is selected from the group consisting of methyl, ethyl, n-propyl, Iso-propyl, n-butyl, iso-butyl, and tert-butyl, 2-hydroxyethyl, 3-hydroxy-n-propyl, 2-hydroxy-isopropyl, 4-hydroxy-n-butyl, 3-hydroxy-isobutyl, and 2-hydroxy-tert-butyl; methyl, ethyl and 2-hydroxyethyl are particularly preferred, and methyl is very particularly preferred.

In a preferred embodiment (variant) the RadP-ROMP monomer has the formula (III):

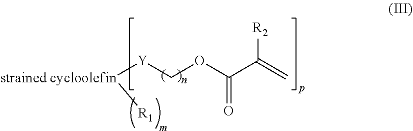

where:
strained cycloolefin is selected from the group consisting of cyclopentene, norbornene, norbornadiene, cyclooctene, tetracyclo[6.2.1.1.0]-dodecene and their oxa derivatives, in which a C atom in the strained cycloolefin structural element is replaced by O. It is preferably selected from the group consisting of norbornene, norbornadiene, tricyclo[5.2.1.0^{2,6}]deca-3,8-diene (dicyclopentadiene), tetracyclo[6.2.1.1.0]-dodecene, and their oxa derivatives, in which a C atom in the strained cycloolefin structural element is replaced by O;
Y is selected from the group consisting of a single bond, —C₁-C₈-alkanediyl-, —O—C₁-C₈-alkanediyl-, —C₁-C₈-alkanediyl-O—. —O—C₁-C₈-alkanediyl-O—, —C₁-C₈-alkanediyl-O—C₁-C₈-alkanediyl-, —C(═O)—O—, —C₁-C₈-alkanediyl-C(═)—O—, —C(═O)—O—C₁-C₈-alkanediyl-, —C(═O)—O—C₁-C₈-alkanediyl-O—C(═O)—, —C₁-C₈-alkanediyl-C(═O)—O—C₁-C₈-alkanediyl-. —O—C(═O)—, —C₁-C₈-alkanediyl-O—C(═O)—, —O—C(═O)—C₁-C₈-alkanediyl-, —O—C(═O)—C₁-C₈-alkanediyl-C(═O)—O—, —C₆-C₁₀-aryldiyl-, —O—C₆-C₁₀-aryldiyl-.
Y is more preferably selected from the group consisting of —C₁-C₈-alkanediyl-, —C₁-C₈-alkanediyl-O—, —C(═O)—O—, —C₁-C₈-alkanediyl-C(═O)—O—, —C(═O)—O—C₁-C₈-alkanediyl-, —O—C(═O)—, —C₁-C₈-alkanediyl-O—C(═O)—.
Y is more preferably selected from the group consisting of —C(═O)—O—, —C₁-C₂-alkanediyl-, —C₁-C₂-alkanediyl-O—, —C(═O)—O—C₁-C₂-alkanediyl-, —C₁-C₂-alkanediyl-C(═O)—O—, —C₁-C₂-alkanediyl-O—C(═O)—. Y is particularly preferably —CH₂—, —CH₂—O—, —C(═O)—O—, —CH₂—C(═O)—O—, —CH_{Y}—O—C(═O)—, or —C(═O)—O—CH₂—. Y is very particularly preferably —CH₂—O—, —C(═O)—O—, —CH₂—C(═O)—O—, or —CH₂—O—C(═O);
m is 0 or 1;
n is an integer from 2 to 6, more preferably 2;
p is an integer from 1 to 2, preferably 1;
R₁ is selected from the group consisting of —C(═O)—OH, —C(═O)—O—C₁-C₄-alkyl, —O—C(═O)—C₁-C₄-alkyl, —C₁-C₄ alkyl, —CH₂—OH, or —CH₂—O—C₁-C₄-alkyl, and preferably is selected from the group consisting of —C(═O)—OH, —C(═O)—O-methyl, —O—C(═O)-methyl, methyl, ethyl, n-propyl, iso-propyl, n-butyl, iso-butyl, and tert-butyl, —CH₂—OH, —CH₂—O— methyl, 2-hydroxyethyl, 3-hydroxy-n-propyl, 2-hydroxy-isopropyl, 4-hydroxy-n-butyl, 3-hydroxy-isobutyl, and 2-hydroxy-tert-butyl. Methyl, ethyl, —CH₂—OH and 2-hydroxyethyl are particularly preferred, and methyl is very particularly preferred. Alternatively, two R₁ together form a bridge of formula —(CH₂)_{z}— which interconnects two cycloolefin structural elements, where z is an integer from 1 to 2, preferably 1, or a bridge of formula —(CH₂)_{q}—C═C—(CH₂)_{q}— which interconnects two cycloolefin structural elements, where q are, independently of one another, an integer from 0 to 1, more preferably both are 0, or a bridge of formula —(CH₂)_{q}O—(CH₂)_{q}— which interconnects two cycloolefin structural elements, where q are, independently of one another, an integer from 0 to 2, preferably from 0 to 1, even more preferably both are 0, or a bridge of formula —C(═O)—O—(CH₂)_{z}—O—(O═)—C— which interconnects two cycloolefin structural elements, where z is an integer from 1 to 4, preferably from 1 to 3, more preferably from 1 to 2, particularly preferably 1, or a bridge of formula —C(═O)—O-phenyl-O—(O═)—C— which interconnects two cycloolefin structural elements, or a bridge of formula —(CH₂)_{q}—Si(R₄)₂—(CH₂)_{q}— which interconnects two cycloolefin structural elements, where q are, Independently of one another, an integer from 0 to 1, more preferably at least once 1, and R₄ is H or a substituted or unsubstituted C₁-C₄ alkyl, more preferably H or an unsubstituted C₁-C₄ alkyl, more preferably an unsubstituted C₁-C₄ alkyl;
R₂ is selected from the group consisting of methyl, ethyl, n-propyl, iso-propyl, n-butyl, iso-butyl, and tert-butyl, 2-hydroxyethyl, 3-hydroxy-n-propyl, 2-hydroxy-isopropyl, 4-hydroxy-n-butyl, 3-hydroxy-isobutyl, and 2-hydroxy-tert-butyl; methyl, ethyl and 2-hydroxyethyl are particularly preferred, and methyl is very particularly preferred.

In a more preferred embodiment, the RadP-ROMP monomer has the following formula (IV), (V), or (VI):

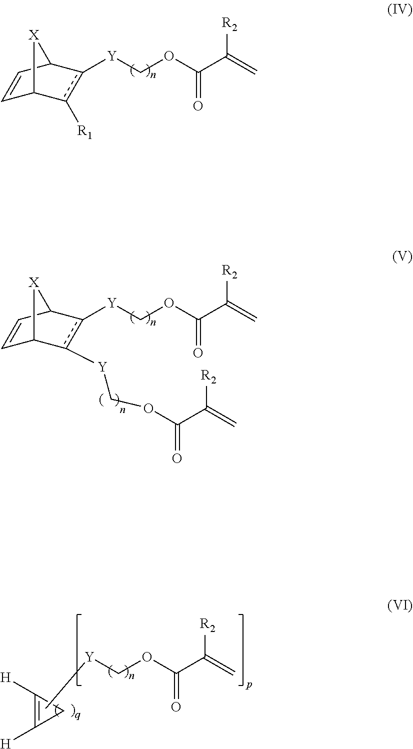

where:
[symbol] is a single or double bond, preferably a single bond;
Y, n, R₁ and p are as defined above for formula (III) and preferably as defined for the preferred variant of formula (III) defined above;
X is —CH₂— or —O—, preferably —CH₂—;
q is an integer selected from the group consisting of 1, 2, 3, 5 and 6, preferably selected from 1, 3 and 5, particularly preferably 3, where p is less than or equal to q.

In an even more preferred embodiment, the RadP-ROMP monomer is selected from the following group consisting of formulas (VII) to (IX):

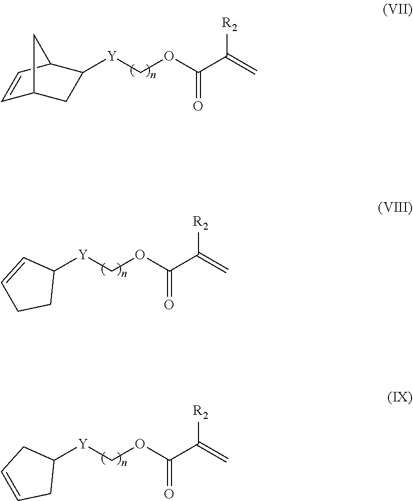

where:
Y, n and R₂ are as defined above for formula (III) and preferably as defined for the preferred variant of formula (III) defined above.

In a particularly preferred embodiment, the RadP-ROMP monomer is a molecule which comprises both a norbornene group and a methacrylate group, i.e. a molecule of formula (VII) in which R₂ is methyl. The RadP-ROMP monomer in this embodiment thus has the following formula (X):

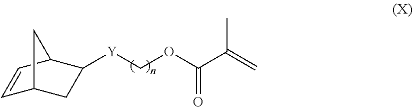

where:
Y and n are as defined above for formula (III) and preferably as defined for the preferred variant of formula (III) defined above.

Most preferably, the RadP-ROMP monomer is a molecule with the formula Mon13 or Mon14, most preferably a molecule with the formula Mon13:

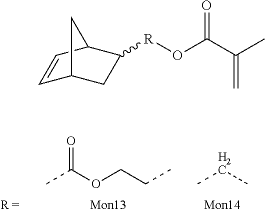

The percentage (in wt.%) of ROMP monomer in component A is advantageously more than approximately 5%, preferably more than approximately 15%, and particularly preferably more than approximately 20%, based on the total weight of component A. The percentage (in wt. %) of ROMP monomer in component A is advantageously approximately 5% to approximately 90%, preferably approximately 8% to approximately 80%, more preferably approximately 10% to approximately 60%, more preferably approximately 20% to approximately 55%, even more preferably approximately 25% to approximately 55%, particularly preferably approximately 25% to approximately 50%, and very particularly preferably approximately 28% to approximately 45%, based on the total weight of component A.

Most preferred are the ROMP monomers or RadP-ROMP monomers used in the examples, preferably in the amounts and/or ratios to the total amount of component A and/or ratios to the total amount of the entire 2C system indicated in the examples.

Radical Initiator

Component A comprises at least one radical initiator. In principle, any radical initiator known to those skilled in the art for the polymerization of methacrylates can be used.

A peroxide is preferably used as the radical initiator. Any of the peroxides known to a person skilled in the art that are used to cure methacrylates can be used. Such peroxides include organic and inorganic peroxides, either liquid or solid, with it also being possible to use hydrogen peroxide. Examples of suitable peroxides are peroxycarbonates (of formula —OC(O)OO—), peroxyesters (of formula —C(O)OO—), diacyl peroxides (of formula —C(O)OOC(O)—), dialkyl peroxides (of formula —OO—), hydroperoxides (of formula —OOH), and the like. These can be present as oligomers or polymers. A comprehensive set of examples of suitable peroxides is described, for example, in the application US 2002/0091214 A1, paragraph [0018].

The peroxides are preferably selected from the group of organic peroxides. Suitable organic peroxides are: tertiary alkyl hydroperoxides such as tert-butyl hydroperoxide and other hydroperoxides such as cumene hydroperoxide, peroxyesters or peracids such as tert-butyl peresters (e.g. tert-butyl peroxybenzoate), benzoyl peroxide (BPO), peracetates and perbenzoates, lauroyl peroxide including (di)peroxyesters, perethers such as peroxy diethyl ether, perketones such as methyl ethyl ketone peroxide. The organic peroxides used as a radical initiator are often tertiary peresters or tertiary hydroperoxides, i.e. peroxide compounds having tertiary carbon atoms which are bonded directly to an —O—O-acyl or —OOH group. However, mixtures of these peroxides with other peroxides can also be used according to the invention. The peroxides may also be mixed peroxides, i.e. peroxides which have two different peroxide-carrying units in one molecule. In a preferred embodiment, benzoyl peroxide (BPO) or tert-butyl peroxybenzoate is used, and BPO is very particularly preferably used.

The peroxide can be used in its pure form, on a carrier material, or as part of a mixture. It is typically used as part of a mixture or on a carrier material, for example on a carrier material such as Perkadox 20S. The peroxide BPO used in the examples and its carrier-bound variant Perkadox 20S are particularly preferred; Perkadox 20S or a comparable carrier-bound BPO is most preferred.

The radical initiator is used in an amount which is tailored to its reactivity. Typically, for example in the case of BPO, this is an amount of from approximately 1.0 wt. % to approximately 25 wt. % based on all monomers with a methacrylate group, more preferably from approximately 2.5 wt. % to approximately 12.5 wt. %, even more preferably from approximately 3.5 wt. % to approximately 7.5 wt. %, even more preferably from approximately 5.0 wt. % to approximately 6.0 wt. %, for example approximately 5.0 wt. % based on all monomers with a methacrylate group. In the case of carrier-bound BPO, the amount used is adjusted according to the corresponding amount of pure BPO. Typically, the amount of the radical initiator for example in the case of Perkadox 20S is therefore an amount of from approximately 5 wt. % to approximately 60 wt. % based on all monomers with a methacrylate group, more preferably from approximately 10 wt. % to approximately 50 wt. %, even more preferably from approximately 20 wt. % to approximately 40 wt %, even more preferably from approximately 25 wt. % to approximately 30 wt. %, for example approximately 30 wt. % based on all monomers with a methacrylate group.

The most preferred radical initiators are the radical initiators used in the examples, preferably in the amounts and/or ratios to the corresponding methacrylate monomers indicated in the examples.

Optional: Filers

In a preferred embodiment, component A also comprises one or more fillers. Additionally, component B can optionally also comprise fillers. In a preferred embodiment, both components comprise fillers, in particular the same fillers, preferably in the same wt. % based on the total amount of the relevant component.

It should be noted that some substances can be used both as a filler and, optionally in modified form, as an additive. For example, fumed silica is preferably used as a filler in the polar, non-after-treated form thereof, and is preferably used as an additive in the non-polar, after-treated form thereof. In cases in which exactly the same substance can be used as a filler or an additive, advantageously the total amount thereof (optionally in sum with other fillers) should not exceed the upper limit for fillers that is established herein.

In order to produce a two-component system for construction applications, in particular for chemical fastening, fillers commonly used for such applications can be added. These fillers are typically inorganic fillers, as described below for example.

The following applies to the calculation of the filler proportion: The proportion of the total fillers (and possibly additives) in the total amount of the relevant component is from 0 wt. % to approximately 90 wt. %, preferably from approximately 20 to approximately 80 wt. %, more preferably from approximately 30 to approximately 70 wt. %, and even more preferably from approximately 50 to approximately 65 wt. %. These wt. % ranges are also the preferred ranges for the proportion of total fillers in the total amount of the 2C system.

The fillers used are conventional fillers, preferably mineral or mineral-like fillers, such as quartz, glass, sand, quartz sand, quartz powder, porcelain, corundum, ceramics, talc, silicic acid (e.g. fumed silica, in particular fumed silica after-treated in a non-polar manner), silicates, aluminum oxides (e.g. alumina), clay, titanium dioxide, chalk, barite, feldspar, basalt, aluminum hydroxide, granite or sandstone, polymeric fillers such as thermosets, hydraulically curable fillers such as gypsum, quicklime or cement (e.g. aluminate cement (often referred to as alumina cement) or Portland cement), metals such as aluminum, carbon black, and also wood, mineral or organic fibers, or the like, or mixtures of two or more thereof.

The fillers may be present in any desired forms, for example as powder or flour, or as shaped bodies, for example in cylindrical, annular, spherical, platelet, rod, saddle or crystal form, or else in fibrous form (fibrillar fillers), and the corresponding base particles preferably have a maximum diameter of approximately 10 mm and a minimum diameter of approximately 1 nm. This means that the diameter is approximately 10 mm or any value less than approximately 10 mm, but more than approximately 1 nm. The maximum diameter is preferably a diameter of approximately 5 mm, more preferably approximately 3 mm, even more preferably approximately 0.7 mm. A maximum diameter of approximately 0.5 mm is very particularly preferred. The more preferred minimum diameter is approximately 10 nm, even more preferably approximately 50 nm, very particularly preferably approximately 100 nm. Diameter ranges resulting from a combination of this maximum diameter and minimum diameter are particularly preferred. However, the globular, inert substances (spherical form) have a preferred and more pronounced reinforcing effect. Core-shell particles, preferably in spherical form, can also be used as fillers.

Preferred fillers are selected from the group consisting of cement, silica, quartz, quartz sand, quartz powder, alumina, corundum and mixtures of two or more thereof. Fillers selected from the group consisting of fumed silica, in particular fumed silica after-treated in a non-polar manner, quartz sand, quartz powder, corundum and mixtures of two or more thereof are particularly preferred. Fillers selected from the group consisting of fumed silica after-treated in a non-polar manner, quartz sand and quartz powder and mixtures of two or more thereof are very particularly preferred. A mixture of a fumed silica after-treated in a non-polar manner, for example a fumed silica after-treated with polydimethylsiloxane (PDMS) (e.g. Cab-O-Sil TS-720), with quartz sand and/or quartz powder is particularly preferred.

If a mixture of a fumed silica after-treated in a non-polar manner, e.g. a fumed silica after-treated with polydimethylsiloxane (PDMS), with quartz sand and/or quartz powder is used, the mixing ratio between fumed silica after-treated in a non-polar manner and the total amount of quartz sand and quartz powder is from approximately 1:30 (w/w) to approximately 1:10 (w/w), preferably from approximately 1:25 (w/w) to approximately 1:15 (w/w), more preferably approximately 1:20. The mixing ratio between fumed silica after-treated in a non-polar manner and the total amount of monomer in each component and in the overall 2C system is advantageously from approximately 1:20 (w/w) to approximately 1:5 (w/w), preferably from approximately 1:15 (w/w) to approximately 1:7.5 (w/w), more preferably from approximately 1:13 (w/w) to 1:9 (w/w), even more preferably from approximately 1:12 (w/w) to approximately 1:10 (w/w).

In this regard, reference is made to the applications WO 02/079341 and WO 02/079293 as well as WO 2011/128061 A1, the contents of which are hereby incorporated in this application.

The mixture of fillers which is used in the examples is very particularly preferred, in particular in the ratios described therein to one another and/or to the total amount of monomers in the 2C system and/or to the amount of monomer in component A.

Component B

Component B comprises a methacrylate (for example methyl methacrylate or BDDMA), a catalyst for ring-opening metathesis polymerization (ROMP initiator), and a radical accelerator (for example DMpT). In a preferred embodiment, component B also comprises an inhibitor (for example Tempol).

(Meth)Acrylate

Component B comprises at least one (meth)acrylate, which is preferably a methacrylate. In the context of the present invention, methacrylates are compounds which contain an esterified methacrylic acid group, such as MMA and BDDMA.

In the context of the present invention, the methacrylates contained in component B are preferably not RadP-ROMP monomers as defined above; if a methacrylate contains a strained cycloolefin structural element and is therefore a RadP-ROMP monomer, it is preferably contained in component A. This applies, for example, to the methacrylates dicyclopentenyloxyethyl methacrylate, tricyclopentadienyl dimethacrylate, dicyclopentenyloxyethyl crotonate, and 3-methylcyclopentadienyl methacrylate.

Suitable methacrylates are aliphatic (linear, branched or cyclic) or aromatic C₅-C₁₅ methacrylates such as methyl methacrylate (MMA), tert-butyl methacrylate and norbomyl methacrylate, as well as 2- or 3-hydroxypropyl methacrylate (HPMA), 1,2-ethanediol dimethacrylate, 1,3-propanediol dimethacrylate, 1,3-butanediol dimethacrylate, 1,4-butanediol dimethacrylate (BDDMA), trimethyloipropane trimethacrylate, phenethyl methacrylate, tetrahydrofurfuryl methacrylate, ethyl triglycol methacrylate, N,N-dimethylaminoethyl methacrylate, N,N-dimethylaminomethyl methacrylate, acetoacetoxyethyl methacrylate, isobornyl methacrylate, 2-ethylhexyl methacrylate, diethylene glycol dimethacrylate, methoxy polyethylene glycol monomethacrylate, trimethylcyclohexyl methacrylate, 2-hydroxyethyl methacrylate (HEMA), bisphenol A methacrylate, novolac epoxy dimethacrylate, di-[methacryloyl-maleoyl]-tricyclo-5.2.1.0.^{2,6}-decane, 3-methacryloyl-oxymethyl-tricyclo-5.2.1.0.^{2,6}-decane, decalyl-2-methacrylate, PEG-dimethacrylate such as PEG200-dimethacrylate, tetraethylene glycol dimethacrylate, solketal methacrylate, cyclohexyl methacrylate, phenoxyethyl dimethacrylate, and methoxyethyl methacrylate.

Preferably, the methacrylate is selected from the group consisting of aliphatic (linear, branched or cyclic) C₅-C₁₅ methacrylates (such as methyl methacrylate (MMA), tert-butyl methacrylate and norbomyl methacrylate), 2-hydroxyethyl methacrylate (HEMA), 2- and 3-hydroxypropyl methacrylate (HPMA), 1,2-ethanediol dimethacrylate, 1,4-butanediol dimethacrylate (BDDMA), 1,3-butanediol dimethacrylate, trimethylolpropane trimethacrylate, acetoacetoxyethyl methacrylate, isobornyl methacrylate, bisphenol A methacrylate, trimethylcyclohexyl methacrylate, 2-hydroxyethyl methacrylate, and PEG200 dimethacrylate. Methyl methacrylate (MMA), tert-butyl methacrylate and norbornyl methacrylate, 2-hydroxyethyl methacrylate (HEMA), 2- and 3-hydroxypropyl methacrylate (HPMA), 1,2-ethanediol dimethacrylate, 1,4-butanediol dimethacrylate (BDDMA) and 1,3-butanediol dimethacrylate are particularly preferred. Methyl methacrylate (MMA), 2-hydroxyethyl methacrylate (HEMA) and 1,4-butanediol dimethacrylate (BDDMA) are very particularly preferred. Most preferred are MMA and BDDMA.

Mixtures of two or more of the methacrylates listed here can also be used.

In addition, the methacrylate may contain, besides the methacrylic acid ester group, other reactive groups that can be polymerized with a radical initiator, such as a peroxide, for example reactive groups derived from itaconic acid, citraconic acid and allylic groups and the like, as described, for example, in WO 2010/108939 A1 (itaconic acid ester).

In addition to the methacrylic acid ester group, the methacrylate can also contain other reactive groups which can be polymerized with a ROMP initiator. In a preferred embodiment, the methacrylate can thus be part of the above-described RadP-ROMP monomer as a methacrylate group (methacrylic acid ester group). In this case, it is not necessary to use a further methacrylate monomer in addition to the ROMP monomer. However, in a preferred embodiment, in addition to the RadP-ROMP monomer, a monomer selected from the group of methacrylates listed above or a mixture of two or more of these monomers is also present in the 2C system according to the invention.

Most preferred are the methacrylates used in the examples, preferably in the amounts and/or ratios to the ROMP monomer (including the optional RadP-ROMP monomer) and/or to the radical initiator which are indicated in the examples.

The methacrylate or methacrylates are preferably present in the 2C system according to the invention in an amount of from 0 to approximately 40 wt. %, particularly preferably from approximately 1 to approximately 30 wt. %, even more preferably from approximately 2 to approximately 25 wt. %, even more preferably from approximately 5 to approximately 20 wt. %, even more preferably from approximately 7 to approximately 15 wt. %, for example approximately 12 wt. % based on the amount of ROMP monomer (with the ROMP monomer here including the optional RadP-ROMP monomer).

If an acrylate is used instead of a methacrylate as the (meth)acrylate, the same applies to such an acrylate as described in the preceding paragraphs for the methacrylate, with the difference that the compound contains an esterified acrylic acid group instead of an esterified methacrylic acid group.

ROMP Initiator

Component B comprises at least one ROMP initiator. In principle, any ROMP catalyst that leads to ring opening in the ring system used and subsequent polymerization can be used as a ROMP initiator for the dual cure process according to the present invention; in other words, any catalyst for ring-opening olefin metathesis polymerization (ROMP). Typically, these are metal-carbene complexes, for example ruthenium-carbene complexes.

In a preferred embodiment, the ROMP initiator used is a Grubbs catalyst. Grubbs catalysts are ruthenium-carbene complexes, are known in the literature and are described, for example, in Grubbs, R. H., Handbook of Metathesis, Wiley-VCH, Germany, 2003: Grubbs, R. H., Trnka, T. M. Ruthenium-catalyzed Olefin Metathesis, in: Ruthenium in Organic Synthesis (S.-I. Murahashi, ed.), Wiley-VCH, Germany, 2004; and Leitgeb, A. et al., Polymer 51(14):2927-2946 and Choi. T. I. and Grubbs, R. H., Angewandte Chemie International Edition 42 (15): 1743-1746. Particularly preferred are Grubbs catalysts with one or more bromopyridine(s) as a ligand, as described e.g. in Walsh, D. J: et al., Journal of the American Chemical Society 139(39):13644-13647, and in particular in Slugovc. C. et al., Macromol. Rapid Commun. 2004(25):475 and J. Mole. Catal. A-Chem, 2004(213):107.

In a preferred embodiment, the Grubbs catalyst has the following formula:

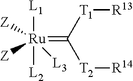

where
Z is Cl, Br, F, I, tosylate or a ligand defined as L₁, L₂, L₃;
L₁, L₂, L₃ are, independently of one another, absent or selected from the group consisting of P(R¹⁵)₃ where R¹⁵=phenyl, isopropyl, cyclohexyl,

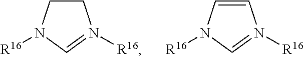

where R¹⁶=mesityl (2,4,6-trimethylphenyl: MES), unsubstituted pyridine, pyridine substituted at position 2 or at positions 2 and 4, the substituents being selected from the group consisting of —Br, —Cl, —F, —I, —OCH₃.
R¹³, R¹⁴ are, independently of one another, selected from the group consisting of H, an aromatic or aliphatic, polycyclic or condensed C₆-C₂₀ ring or C₆-C₂₀ ring system, with 1-5 heteroatoms in the ring, independently selected from N, S, O, and P, the ring or ring system being unsubstituted or additionally substituted by 1-5 substituents which, independently of one another, are selected from the group consisting of —Cl, —Br, —I, —F, —OR¹⁷, —CH═N—R¹⁷, —C(═O)R¹⁷, —C(═O)OR¹⁷, —OC(═O)R¹⁷, where R¹⁷ is a linear or branched acyclic C₁-C₁₄ alkyl or cyclic C₃-C₁₄ alkyl or a C₆-C₁₄ aryl, where R¹⁷ can contain 1-5 heteroatoms, selected independently of one another from the group consisting of N, O, Si, and S;
T₁, T₂ are, independently of one another, absent or selected from the group consisting of —O—, —S—, saturated C₁-C₄ alkylene, and unsaturated C₂-C₄ alkylene, where the alkylene can contain 0-2 heteroatoms selected independently of one another from the group consisting of N, O, S, and Si.

The Grubbs Catalyst Used as a ROMP Initiator is Preferably Selected from the Group Consisting of:

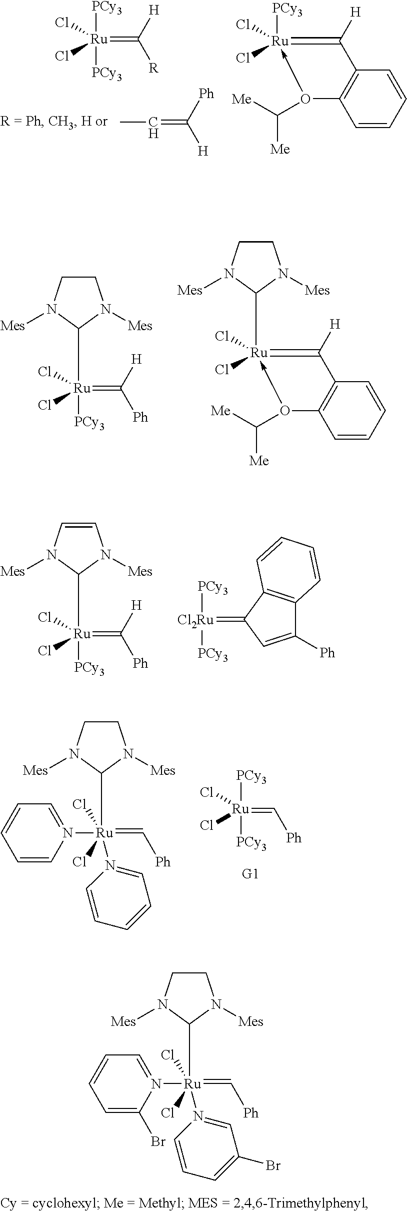

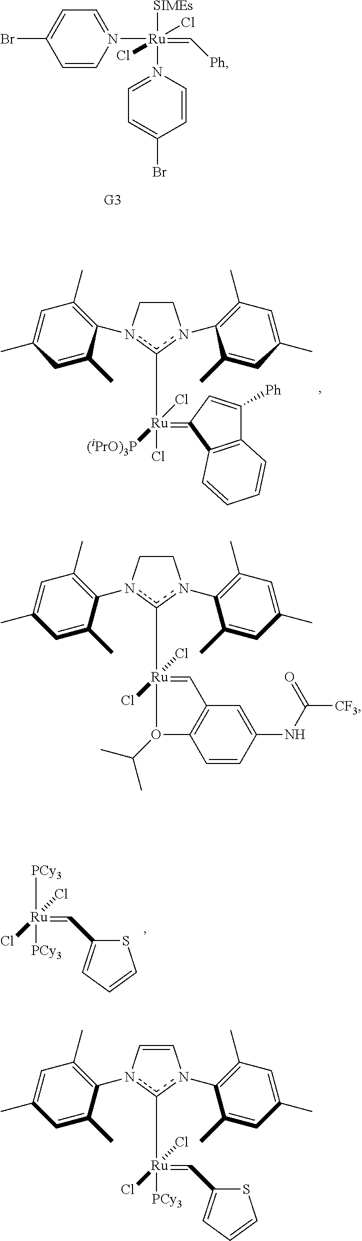

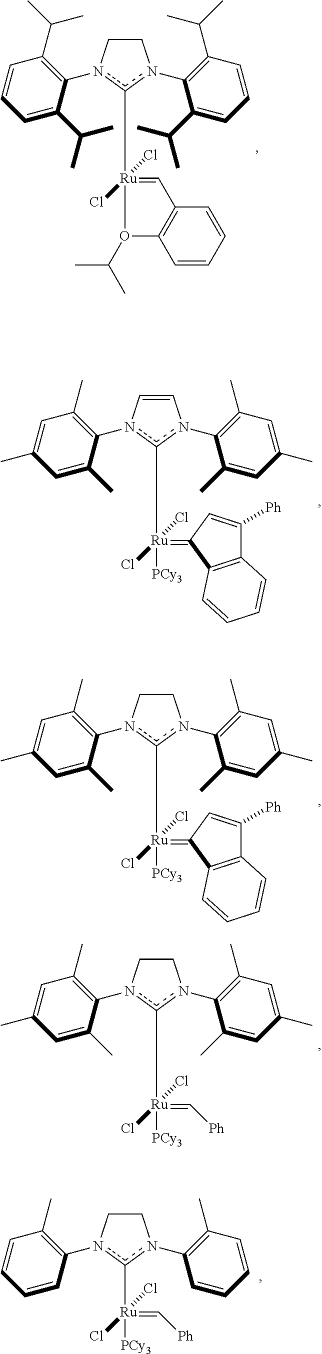

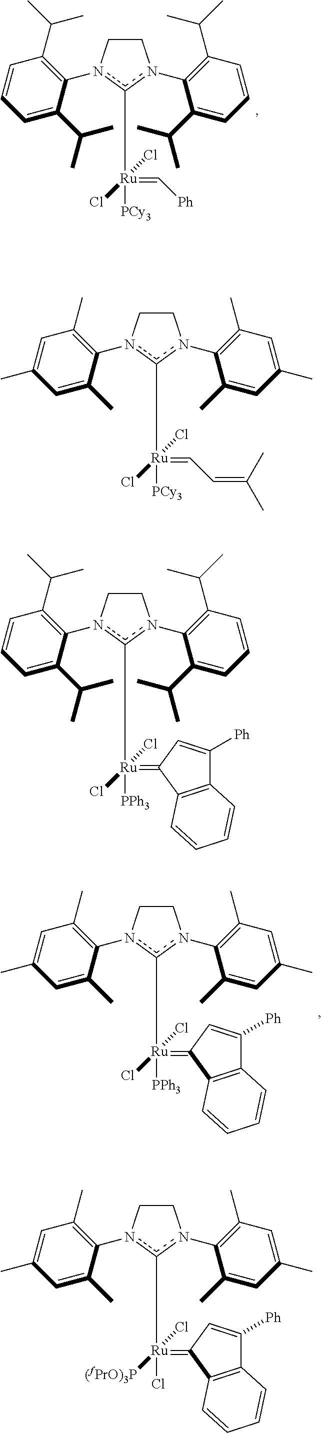

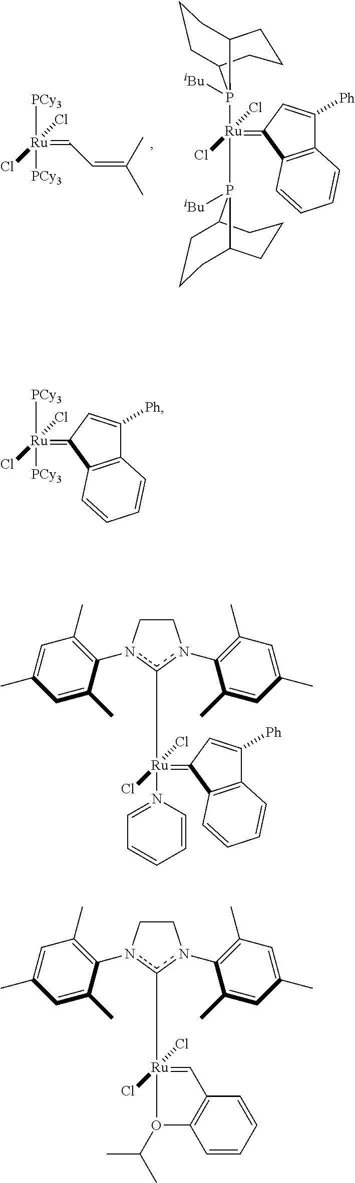

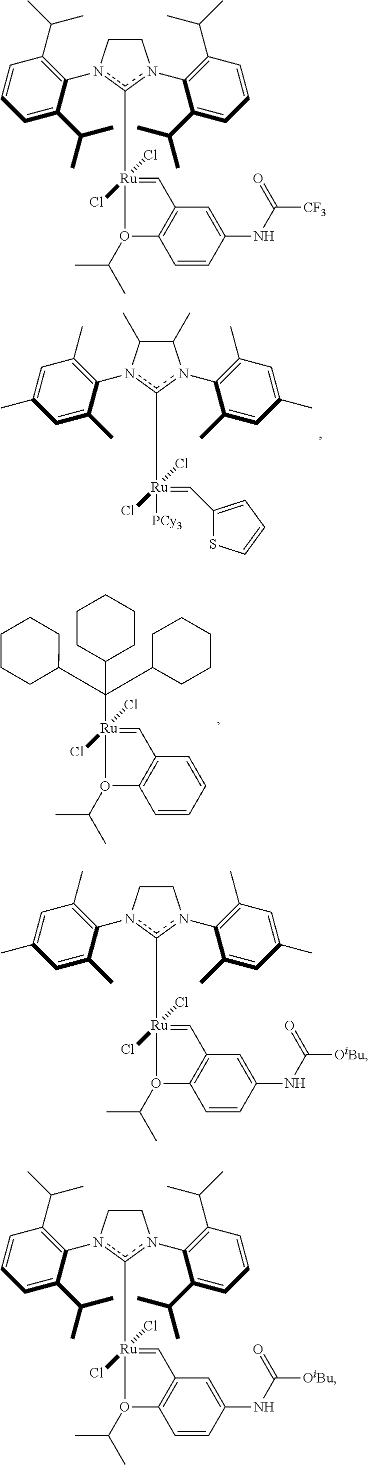

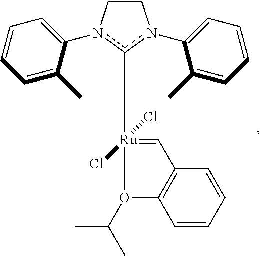

[1,3-bis[1,3-bis(2,4,6-trimethylphenyl)-2-imidazolidinylidene]-dichloro-(3-phenyl-1H-inden-1-ylidene)ruthenium(II), bis(2,4,6-trimethylphenyl)-2-imidazolidinylidene]dichloro-(3-phenyl-1H-inden-1-ylidene)(triphenylphosphine)ruthenium(II), and [1,3-bis(2,4,6-trimethylphenyl)-2-imidazolidinylidene]-dichloro-(3-phenyl-1H-inden-1-ylidene)(pyridyl)ruthenium(II).

In one embodiment of the present invention, the Grubbs catalyst is the complex G3, the complex M20, i.e. [1,3-bis(2,4,6-timethylphenyl)-2-imidazolidinylidene]-dichloro-(3-phenyl-1H-inden-1-ylidene)(triphenylphosphine)ruthenium(II), the complex M31, i.e. [1,3-bis(2,4,6-trimethylphenyl)-2-imidazolidinylidene]-dichloro-(3-phenyl-1H-indene-1-ylidene)(pyridyl)ruthenium(II), or the Hoveyda Grubbs catalyst M722, all available, for example, from UMICORE AG & Co. KG. The Grubbs catalyst M20, M31, or M722 is preferred, and the most preferred ROMP initiator is M20.

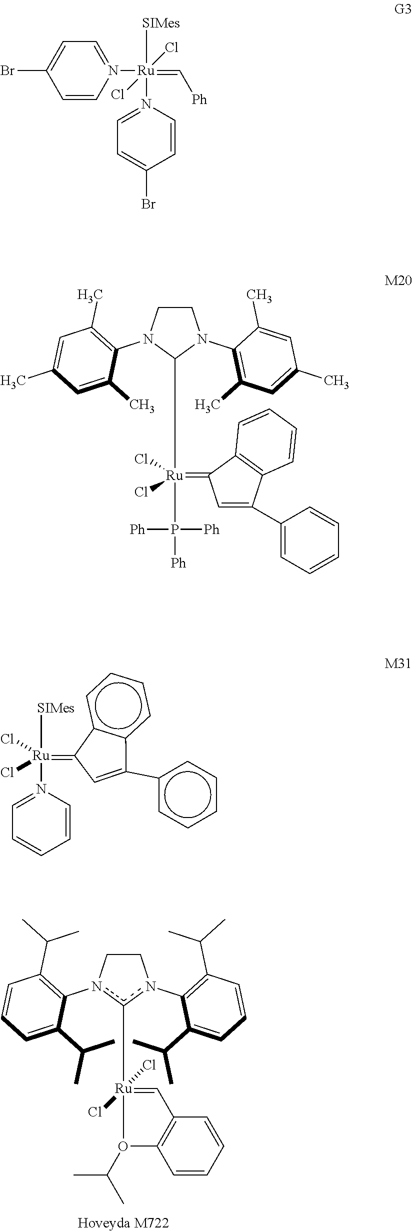

Combinations of two or more ROMP initiators can also be used.

In the two-component system according to the invention, the ROMP initiator is advantageously separate from the radical initiator. This is because the radical initiator can reduce the storage stability of the ROMP initiator. Furthermore, the ROMP initiator in the two-component system according to the invention is advantageously present in the same component as the radical accelerator, in particular if the radical accelerator is one of the (preferred) amines mentioned here as radical accelerators. This can increase the storage stability of the ROMP initiator. In a preferred embodiment, this combination is realized by component B according to the invention.

The ROMP initiator in the 2C system according to the invention is typically in an amount of from approximately 10 ppm to approximately 500 ppm, preferably from approximately 20 ppm to approximately 300 ppm, more preferably from approximately 30 ppm to approximately 100 ppm, even more preferably from approximately 40 ppm to approximately 70 ppm, for example approximately 50 ppm, based on the total amount of ROMP monomer.

The most preferred ROMP initiators are the ROMP initiators used in the examples, preferably in the amounts and/or ratios to the corresponding ROMP monomers indicated in the examples.

Radical Accelerator

Component B further comprises at least one radical accelerator. Suitable accelerators for use in combination with a radical initiator are known to a person skilled in the art. Suitable radical accelerators in the context of the present application are expediently organic amines.

Organic amines suitable as radical accelerators are selected from among the following compounds, which are described, for example, in the application US 2011/071234 A1: Dimethylamine, trimethylamine, ethylamine, diethylamine, triethylamine, n-propylamine, di-n-propylamine, tri-n-propylamine, iso-propylamine, di-iso-propylamine, tri-iso-propylamine, n-butylamine, iso-butylamine, tert-butylamine, di-n-butylamine, di-iso-butylamine, tri-iso-butylamine, n-pentylamine, iso-pentylamine, di-iso-pentylamine, hexylamine, octylamine, dodecylamine, laurylamine, stearylamine, aminoethanol, diethanolamine, triethanolamine, aminohexanol, ethoxyaminoethane, dimethyl(2-chloroethyl)amine, 2-ethylhexylamine, bis(2-chloroethyl)amine, 2-ethylhexylamine, bis(2-ethylhexyl)amine, N-methylstearylamine, dialkylamines, ethylenediamine. N,N′-dimethylethylenediamine, tetramethylethylenediamine, diethylenetriamine, permethyldiethylenetriamine, triethylenetetramine, tetraethylenepentamine, 1,2-diaminopropane, di-propylenetriamine, tripropylenetetramine, 1,4-diaminobutane, 1,6-diaminohexane, 4-amino-1-diethylaminopentane, 2,5-diamino-2,5-dimethylhexane, trimethylhexamethylenediamine, N,N-dimethylaminoethanol, 2-(2-diethylaminoethoxy)ethanol, bis(2-hydroxyethyl)oleylamine, tris[2-(2-hydroxyethoxy)ethyl]amine, 3-amino-1-propanol, methyl(3-aminopropyl)ether, ethyl-(3-aminopropyl)ether, 1,4-butanediol-bis(3-aminopropyl ether), 3-dimethylamino-1-propanol, 1-amino-2-propanol, 1-diethylamino-2-propanol, di-iso-propanolamine, methyl-bis(2-hydroxypropyl)amine, tris(2-hydroxypropyl)amine, 4-amino-2-butanol, 2-amino-2-methylpropanol, 2-amino-2-methylpropanediol, 2-amino-2-hydroxymethylpropanediol, 5-diethylamino-2-pentanone, 3-methylaminopropionitrile, 6-aminohexanoic acid, 11-aminoundecanoic acid, 6-aminohexanoic acid ethyl ester, 11-aminohexanoate-isopropyl ester, cyclohexylamine, N-methylcyclohexylamine. N,N-dimethylcyclohexylamine, dicyclohexylamine, N-ethylcyclohexylamine, N-(2-hydroxyethyl)cyclohexylamine, N,N-bis(2-hydroxyethyl)cyclohexylamine, N-(3-aminopropyl)cyclohexylamine, aminomethylcyclohexane, hexahydrotoluidine, hexahydrobenzylamine, aniline. N-methylaniline, N,N-dimethylaniline, N,N-diethylaniline, N,N-di-propylaniline, iso-butylaniline, toluidines, diphenylamine, hydroxyethylaniline, bis(hydroxyethyl)aniline, chloroaniline, aminophenols, aminobenzoic acids and esters thereof, benzylamine, dibenzylamine, tribenzylamine, methyidibenzylamine, β-phenylethylamine, xylidine, di-iso-propylaniline, dodecylaniline, aminonaphthalene, N-methylaminonaphthalene, N,N-dimethylamino-naphthalene, N,N-dibenzylnaphthalene, diaminocyclohexane, 4,4′-diamino-dicyclohexyl-methane, diamino-dimethyl-dicyclohexylmethane, phenylenediamine, xylylenediamine, diaminobiphenyl, naphthalenediamines, toluidines, benzidines, 2,2-bis(aminophenyl)propane, aminoanisoles, aminothiophenols, aminodiphenyl ethers, aminocresols, morpholine, N-methylmorpholine, N-phenylmorpholine, hydroxyethyl-morpholine, N-methylpyrrolidine, pyrrolidine, piperidine, hydroxyethylpiperidine, pyrroles, N,N-bisalkylarylamines, pyridines, quinolines, indoles, indolenines, carbazoles, pyrazoles, imidazoles, thiazoles, pyrimidines, quinoxalines, aminomorpholine, dimorpholineethane, and [2,2,2]-diazabicyclooctane.

Particularly preferred within the context of the present invention are aromatic amines, most preferably anilines, toluidines and N,N-bisalkylarylamines such as N,N-dimethylaniline, N,N-diethylaniline, N,N-di-iso-propanol-p-toluidine (DiPpT). N,N-di-hydroxyethyl-m-toluidine (DHEmT). N,N-dimethyl-p-toluidine (DMpT), N,N-diethyl-p-toluidine (DEpT), N,N-bis(hydroxyalkyl)arylamines, N,N-bis(2-hydroxyethyl)anilines, N,N-bis(2-hydroxyethyl)-toluidine, N,N-bis-(2-hydroxypropyl) aniline, N,N-bis(3-methacryloyl-2-hydroxypropyl)-p-toluidine, N,N-dibutoxyhydroxypropyl-p-toluidine and 4,4′-bis(dimethylamino)diphenylmethane. In particular. N,N-di-iso-propanol-p-toluidine (DiPpT). N,N-di-hydroxyethyl-m-toluidine (DHEmT), N,N-dimethyl-p-toluidine (DMpT) and N,N-diethyl-p-toluidine (DEpT) are preferred. DiPpT and DMpT are very particularly preferred, and DMpT is most preferred.

A single radical accelerator or a mixture of multiple radical accelerators can be used. The use of a single radical accelerator is preferred.

The radical accelerator is typically used in an amount of from approximately 0.01 to approximately 10 wt. %, preferably from approximately 0.1 to approximately 5 wt. %, more preferably from approximately 0.2 to approximately 3 wt. %, even more preferably from approximately 0.4 to approximately 2.0 wt. %, even more preferably from approximately 0.5 to approximately 1.5 wt. %, even more preferably from approximately 0.5 to approximately 1.3 wt. %, and even more preferably from approximately 0.8 to 1.2 wt.%, based on the ROMP monomer (with the ROMP monomer optionally including the RadP-ROMP monomer).

The radical accelerator must match the radical initiator used. Which pairings these are and in what proportions they can be used in relation to each other is known to a person skilled in the art. The amount ratio between the radical initiator and radical accelerator is preferably such that radical polymerization is complete in a time that is advantageous for chemical anchors.

In a preferred embodiment, the radical initiator is a peroxide, preferably BPO. Typically, the weight ratio of radical accelerator (which is preferably DMpT) to peroxide is from 1:1 to 1:10, preferably from 1:3 to 1:8, more preferably from 1:5 to 1:7, for example DMpT:BPO 1:6. In the case of carrier-bound BPO, the amount used is adjusted according to the corresponding amount of pure BPO. Typically, the weight ratio of radical accelerator (which is preferably DMpT) to Perkadox 20S is therefore from 1:5 to 1:50, preferably from 1:15 to 1:40, more preferably from 1:25 to 1:35, for example DMpT:Perkadox 20S 1:30.

The most preferred radical accelerators are the radical accelerators used in the examples, preferably approximately in the amounts and/or ratios to the corresponding radical initiators indicated in the examples.

Optional: Inhibitor

One or more inhibitors are preferably present in the two-component system according to the invention, both for stabilization and for adjusting the reactivity. The inhibitor or inhibitors can be contained in component A or component B; it is/they are preferably contained in component B only.

The inhibitors usually used for radically polymerizable compounds, as are known to a person skilled in the art, are suitable for this purpose. These inhibitors are preferably selected from phenolic inhibitors and non-phenolic inhibitors, in particular phenothiazines.

Phenols such as 2-methoxyphenol, 4-methoxyphenol, 2,6-di-tert-butyl-4-methylphenol, 2,4-di-tert-butylphenol, 2,6-di-tert-butylphenol, 2,4,6-trimethylphenol, 2,4,6-tris(dimethylaminomethyl)phenol, 4,4′-thio-bis(3-methyl-6-tert-butylphenol), 4,4′-isopropylidenediphenol, 6,6′-di-tert-butyl-4,4′-bis(2,6-di-tert-butylphenol), 1,3,5-trimethyl-2,4,6-tris(3,5-di-tert-butyl-4-hydroxybenzyl)benzene, 2,2′-methylene-di-p-cresol, catechols such as pyrocatechol, and catechol derivatives such as butylpyrocatechols such as 4-tert-butylpyrocatechol and 4,6-di-tert-butylpyrocatechol, hydroquinones such as hydroquinone, 2-methythydroquinone, 2-tert-butylhydroquinone, 2,5-di-tert-butylhydroquinone, 2,6-di-tert-butythydroquinone, 2,6-dimethylhydroquinone, 2,3,5-trimethylhydroquinone, benzoquinone, 2,3,5,6-tetrachloro-1,4-benzoquinone, methylbenzoquinone, 2,6-dimethylbenzoquinone, naphthoquinone, or mixtures of two or more thereof, are suitable as phenolic inhibitors. These inhibitors are often part of commercial two-component radically curing systems.

Phenothiazines such as phenothiazine and/or derivatives or combinations thereof, or stable organic radicals such as galvinoxyl and N-oxyl radicals, in particular of the piperidinyl-N-oxyl or tetrahydropyrrole-N-oxyl type, are preferably considered as non-phenolic inhibitors, such as aluminum-N-nitrosophenylhydroxylamine, diethylhydroxylamine, oximes such as acetaldoxime, acetone oxime, methyl ethyl ketoxime, salicyloxime, benzoxime, glyoximes, dimethylglyoxime, acetone-O-(benzyloxycarbonyl)oxime, TEMPOL. TEMPO and the like.

Furthermore, pyrimidinol or pyridinol compounds substituted in para-position to the hydroxyl group, as described in patent DE 10 2011 077 248 B1, can be used as inhibitors.

Stable N-oxyl radicals that can be used, for example, are those described in DE 199 56 509 A1 and DE 195 31 649 A1. Stable N-oxyl radicals of this kind are of the piperidinyl-N-oxyl or tetrahydropyrrole-N-oxyl type or a mixture thereof.

Preferred stable N-oxyl radicals are selected from the group consisting of 1-oxyl-2,2,6,6-tetramethylpiperidine, 1-oxyl-2,2,6,6-tetramethylpiperidin-4-ol (also referred to as 4-hydroxy-2,2,6,6-tetramethylpiperidinyloxyl or TEMPOL), 1-oxyl-2,2,6,6-tetramethylpiperidin-4-one (also referred to as TEMPON), 1-oxyl-2,2,6,6-tetramethyl-4-carboxyl-piperidine (also referred to as 4-carboxy-TEMPO), 1-oxyl-2,2,5,5-tetramethylpyrrolidine, 1-oxyl-2,2,5,5-tetramethyl-3-carboxylpyrrolidine (also referred to as 3-carboxy-PROXYL) and mixtures of two or more of these compounds, with 1-oxyl-2,2,6,6-tetramethylpiperidin-4-ol (synonym: 4-hydroxy-2,2,6,6-tetramethylpiperidinyloxyl or TEMPOL) being particularly preferred.

The inhibitor or inhibitors are preferably selected from the group consisting of stable N-oxyl radicals, catechols, catechol derivatives and phenothiazines and a mixture of two or more thereof. The inhibitor or inhibitors selected from the group consisting of Tempol, catechols and phenothiazines are particularly preferred. The inhibitor TEMPOL is even more preferred; in the case of a mixture of inhibitors, one of them is preferably TEMPOL.

The inhibitors used in the examples are very particularly preferred, preferably in the amounts and ratios to the ROMP monomer and/or to the radical initiator indicated in the examples.

The inhibitors can be used either alone or as a combination of two or more thereof, depending on the desired properties of the two-component system. The combination of phenolic and non-phenolic inhibitors is preferred when a combination of inhibitors is used.

The inhibitor or the inhibitor mixture is added in the usual amounts known in the art, preferably in an amount of from approximately 0.01 to approximately 2 wt. %, more preferably from approximately 0.05 to approximately 1% wt. %, more preferably from approximately 0.1 to approximately 1% wt. %, more preferably from approximately 0.15 to approximately 0.5% wt. %, for example 0.23% wt. % or 0.35 wt. %, based on the total amount of the ROMP monomer (with the ROMP monomer optionally including the RadP-ROMP monomer).

Further Optional Ingredients of the 2C System

The two-component system according to the invention can optionally also comprise further ingredients in addition to the ingredients already mentioned. All optional ingredients commonly used in 2C systems are possible as such optional ingredients, such as dyes, additives (for example thickeners or antioxidants), and reactive diluents.

Reactive Diluent

One or both components of the two-component system can contain at least one reactive diluent in addition to the above-described obligatory monomers in components A and B.

A reactive diluent is preferably contained only in component B. However, component A can also contain a reactive diluent, provided it is a reactive diluent whose simultaneous presence with the radical initiator in component A does not initiate a radical reaction. This is the case, for example, for allyl ethers and vinyl ethers, which participate as monomers in the co-polymerization started by mixing only after mixing with the methacrylate-containing component B.

Suitable reactive diluents are low-viscosity, radically co-polymerizable compounds, preferably compounds free of labeling. Such compounds are known in the art.

Particularly suitable reactive diluents are the methacrylic acid esters already mentioned above. They are already mandatory in component B in any case.

In principle, other conventional radically polymerizable compounds, alone or in a mixture with the methacrylic acid esters, can also be used as reactive diluents, e.g. styrene, α-methylstyrene, alkylated styrenes, such as tert-butylstyrene, divinylbenzene and vinyl and allyl compounds, of which the representatives that are not subject to labeling are preferred. Examples of vinyl or allyl compounds of this kind are vinyl ether or ester and allyl ether or ester, for example hydroxybutyl vinyl ether, ethylene glycol divinyl ether, 1,4-butanediol divinyl ether, trimethylolpropane divinyl ether, trimethyloipropane trivinyl ether, mono-, di-, tri-, tetra- and polyalkylene glycol vinyl ether, mono-, di-, tri-, tetra- and polyalkylene glycol allyl ether, adipic acid divinyl ester, trimethylolpropane diallyl ether and trimethylolpropane triallyl ether.

The reactive diluent(s) is/are preferably present in the 2C system in an amount of from 0 to approximately 30 wt. %, particularly preferably from approximately 0 to approximately 20 wt. %, even more preferably from approximately 5 to approximately 15 wt. %, based on the ROMP monomer. Most preferably, no reactive diluent is present in addition to the methacrylate contained in component B according to the invention.

Additives

Conventional additives are used as the additives, i.e. thixotropic agents, such as optionally organically or inorganically after-treated fumed silica (if not already used as a filler), in particular fumed silica after-treated in a non-polar manner (which in the context of the present invention has already been described in more detail above in connection with the fillers), bentonites, alkyl- and methylcelluloses, castor oil derivatives or the like, plasticizers, such as phthalic or sebacic acid esters, antistatic agents, thickeners, antioxidants, flexibilizers, rheology aids, wetting agents, coloring additives, such as dyes or in particular pigments, for example for different staining of the components for improved control of the mixing thereof, or the like, or mixtures of two or more thereof. Non-reactive diluents (solvents) can also be present, preferably in an amount of up to 30 wt. %, based on the total amount of the component, such as low-alkyl ketones, for example acetone, di-low-alkyl low-alkanoyl amides, such as dimethylacetamide, low-alkylbenzenes, such as xylenes or toluene, phthalic acid esters or paraffins, water or glycols. Furthermore, metal scavengers in the form of surface-modified fumed silicas can be present. Preferably, at least one thixotropic agent is present as an additive, particularly preferably an organically or inorganically after-treated fumed silica, very particularly preferably a fumed silica after-treated in a non-polar manner, e.g. fumed silica after-treated with polydimethylsiloxane (PDMS). In this regard, reference is made to the applications WO 02/079341 and WO 02/079293 as well as WO 2011/128061 A1, the contents of which are hereby incorporated in this application.

Adhesion Promoter

In one embodiment, the two-component system can additionally contain an adhesion promoter. By using an adhesion promoter, the cross-linking of the borehole wall with the anchor mass is improved such that the adhesion increases in the cured state. This is important for the use of a two-component anchor mass, for example in boreholes drilled with a diamond drill, and increases the failure bond strength. Suitable adhesion promoters are selected from the group of silanes which are functionalized with further reactive organic groups and can be incorporated into the polymer network. This group includes, for example, 3-(meth)acryloyloxypropyltrimethoxysilane, 3-(meth)acryloyloxypropyltriethoxysilane, 3-(meth)acryloyloxymethyltrimethoxysilane, 3-(meth)acryloyloxymethyltriethoxysilane, vinyltrimethoxysilane, vinyltriethoxysilane, functionalized tetraethoxysilane, functionalized tetramethoxysilane, functionalized tetrapropoxysilane, functionalized ethyl or propyl polysilicate, and mixtures of two or more thereof. In this regard, reference is made to application DE 10 2009 059210, the content of which is incorporated herein by reference.

The adhesion promoter is expediently contained in amounts of from approximately 1 to approximately 10 wt. %, based on the total weight of the relevant component.

Two-Component System

The system according to the invention is typically a two-component system. However, by dividing the ingredients, it can also be packaged as a multi-component system with more than two components, e.g. three components. These three or more components are then mixed together at the same time or one after the other to start the polymerization. However, a two-component system as described herein is preferred.

If a multi-component system is used, the radical initiator, as already described herein, should advantageously be in a different component than the ROMP initiator. Furthermore, the radical initiator must be in a different component than the methacrylate, and the ROMP initiator must be in a different component than the ROMP monomer.

In one embodiment of the 2C system according to the invention, component A contains:
the ROMP monomer as defined above, preferably a preferred embodiment thereof as described above; and
at least one radical initiator as defined above, preferably a preferred embodiment thereof as described above:
   and component B contains:
the methacrylate as defined above, preferably a preferred embodiment thereof as described above;
at least one ROMP initiator as defined above, preferably a preferred embodiment thereof as described above; and
at least one radical accelerator as defined above, preferably a preferred embodiment thereof as described above.

In a preferred embodiment of the 2C system according to the invention, component A contains:
the ROMP monomer as defined above, preferably a preferred embodiment thereof as described above;
at least one radical initiator as defined above, preferably a preferred embodiment thereof as described above; and
at least one filler as defined above, preferably a preferred embodiment thereof as described above;
   and component B contains:
the methacrylate as defined above, preferably a preferred embodiment thereof as described above;
at least one ROMP initiator as defined above, preferably a preferred embodiment thereof as described above;
at least one radical accelerator as defined above, preferably a preferred embodiment thereof as described above; and
at least one inhibitor as defined above, preferably a preferred embodiment thereof as described above.

In a more preferred embodiment of the 2C system according to the invention, component A contains:
the ROMP monomer as defined above, preferably a preferred embodiment thereof as described above;
at least one radical initiator as defined above, preferably a preferred embodiment thereof as described above; and
at least one filler as defined above, preferably a preferred embodiment thereof as described above;
   and component B contains:
the methacrylate as defined above, preferably a preferred embodiment thereof as described above;
at least one ROMP initiator as defined above, preferably a preferred embodiment thereof as described above;
at least one radical accelerator as defined above, preferably a preferred embodiment thereof as described above;
at least one inhibitor as defined above, preferably a preferred embodiment thereof as described above; and
at least one filler as defined above, preferably a preferred embodiment thereof as described above.

In a further embodiment of the 2C system according to the invention, component A contains:
the RadP-ROMP monomer as defined above, preferably a preferred embodiment thereof as described above; and
at least one radical initiator as defined above, preferably a preferred embodiment thereof as described above;
   and component B contains:
at least one ROMP initiator as defined above, preferably a preferred embodiment thereof as described above; and
at least one radical accelerator as defined above, preferably a preferred embodiment thereof as described above.

In a more preferred further embodiment of the 2C system according to the invention, component A contains:
the RadP-ROMP monomer as defined above, preferably a preferred embodiment thereof as described above;
at least one radical initiator as defined above, preferably a preferred embodiment thereof as described above; and
   and component B contains:
the methacrylate as defined above, preferably a preferred embodiment thereof as described above;
at least one ROMP initiator as defined above, preferably a preferred embodiment thereof as described above;
at least one radical accelerator as defined above, preferably a preferred embodiment thereof as described above; and
at least one inhibitor as defined above, preferably a preferred embodiment thereof as described above.

In a more preferred further embodiment of the 2C system according to the invention, component A contains:
the RadP-ROMP monomer as defined above, preferably a preferred embodiment thereof as described above;
at least one radical initiator as defined above, preferably a preferred embodiment thereof as described above; and
at least one filler as defined above, preferably a preferred embodiment thereof as described above;
   and component B contains:
the methacrylate as defined above, preferably a preferred embodiment thereof as described above;
at least one ROMP initiator as defined above, preferably a preferred embodiment thereof as described above;
at least one radical accelerator as defined above, preferably a preferred embodiment thereof as described above; and
at least one inhibitor as defined above, preferably a preferred embodiment thereof as described above.

In a more preferred further embodiment of the 2C system according to the invention, component A contains:
the RadP-ROMP monomer as defined above, preferably a preferred embodiment thereof as described above;
at least one radical initiator as defined above, preferably a preferred embodiment thereof as described above; and
at least one filler as defined above, preferably a preferred embodiment thereof as described above;
   and component B contains:
the methacrylate as defined above, preferably a preferred embodiment thereof as described above;
at least one ROMP initiator as defined above, preferably a preferred embodiment thereof as described above;
at least one radical accelerator as defined above, preferably a preferred embodiment thereof as described above;
at least one inhibitor as defined above, preferably a preferred embodiment thereof as described above; and
at least one filler as defined above, preferably a preferred embodiment thereof as described above.

In addition, one or more further ROMP monomers can be present in component A and/or one or more further methacrylates can be present in component B.

Components A and components B from each of these embodiments can be combined with one another in any combination A+B to form a 2C system.

In a particularly preferred embodiment, the constituents of the 2C system are one or more of the constituents which are mentioned in the examples according to the invention, 2C systems which contain the same constituents or consist of the same constituents as are mentioned in the individual examples according to the invention, preferably approximately in the proportions stated in said examples, are very particularly preferred.

In a very particularly preferred embodiment of the 2C system according to the invention, the 2C system contains the constituents of components A and/or B, preferably A and B, as described in Example 4 or 5, preferably in Example 5, for these components. Most preferably, the 2C system contains components A and/or B, preferably A and B, as described in Example 4 or 5, preferably in Example 5, for these components, in approximately the proportions as described in these examples.

### Use of the Two-Component System as a Chemical Anchor

The 2C systems according to the invention can be used in many fields in which methacrylates are usually used.

However, the present invention relates in particular to the use of a two-component system described here for the chemical fastening of an anchoring means in a borehole, i.e. as a chemical anchor. In a preferred embodiment of the two-component system, component A contains, in addition to the monomer and the ROMP initiator, a filler as described herein. This filler increases the strength of the chemical anchor in the borehole.

The two-component system comprises component A and component B, separated in different containers in a reaction-inhibiting manner, for example a multi-chamber device such as a multi-chamber cartridge, from which containers the two components are ejected by the application of mechanical ejection forces or by the application of a gas pressure and are mixed. Another possibility is to produce the two-component system as two-component capsules which are introduced into the borehole and are destroyed by placement of the fixing element in a rotational manner, while simultaneously mixing the two components of the mortar composition. A cartridge system or an injection system is preferably used in which the two components are ejected out of the separate containers and passed through a static mixer in which they are homogeneously mixed and then discharged through a nozzle preferably directly into the borehole.

The 2C system according to the invention can be used in the form of a cartridge system or a film pouch system. In the intended use of the system, the components are either ejected from the cartridges or film pouches under the application of mechanical forces or by gas pressure, and are mixed together, preferably by means of a static mixer through which the constituents are passed. The resulting mixture can then be used as a fastening means or for some other purpose. If the 2C system is used as a chemical anchor, the mixture is introduced into e.g. a borehole immediately after mixing, after which the devices to be fastened, such as threaded anchor rods and the like, are introduced into the borehole provided with the curing mixture and adjusted accordingly.

The two-component system according to the invention typically contains component A and component 8 in a weight ratio of from approximately 1:1 to approximately 20:1. The two-component system according to the invention preferably contains component A and component B in a weight ratio of from approximately 3:1 to approximately 15:1, more preferably from approximately 4:1 to approximately 12:1. The two-component system according to the invention particularly preferably contains component A and component B in a weight ratio of approximately 10:1. The same weight ratio ranges are preferred for the ratio of ROMP monomer to methacrylate.

The 2C system according to the invention is used primarily in the construction sector, for example for the repair of concrete, as polymer concrete, as a coating composition or as a cold-curing road marking. Said system is particularly suitable for chemically fastening anchoring means, such as anchors, threaded rods, reinforcing bars, screws and the like, in boreholes, in particular in boreholes in various substrates, in particular mineral substrates, such as those based on concrete, aerated concrete, brickwork, limestone, sandstone, natural stone, glass and the like, and metal substrates such as those made of steel. In one embodiment, the substrate of the borehole is concrete, and the anchoring means consists of steel or iron. In a further embodiment, the substrate of the borehole is steel, and the anchoring means consists of steel or iron.

The invention is explained in greater detail in the following with reference to a number of examples. However, the invention is not limited to the specific embodiments shown in the examples.

### EXAMPLES

All chemicals and constituents of the compositions listed here are—unless stated otherwise—commercially available and were used in the commercially usual quality.

The complex M20, i.e. [1,3-bis(2,4,6-trimethylphenyl)-2-imidazolidinylidene]-dichloro-(3-phenyl-1H-inden-1-ylidene)(triphenylphosphine)ruthenium(II), used as a ROMP initiator, was from UMICORE AG & Co. KG.

Perkadox 20S consists of 20 wt. % BPO on inert fillers and is available from Akzo Nobel, The Netherlands.

Unless stated otherwise, all % and ppm data given in the examples in connection with amounts relate to the total weight of the composition described, as a calculation basis.

Silica gel 60 F254 on aluminum was used for thin-layer chromatography (Merck).

Visualization with UV light (254 nm) unless stated otherwise.

Silica gel 60 (220-440 mesh ASTM) was used for column chromatography.

¹H- and ¹³C-NMR measurements were carried out at 25° C. on a Bruker Avarice 300 MHz (¹H: 300.36 MHz, ¹³C: 75.53 MHz). The ppm values are relative to tetramethylsilane (TMS).

### Example 1: Synthesis of Norbornene-Containing Monomers

### Example 1.1: Diels-Alter Reaction

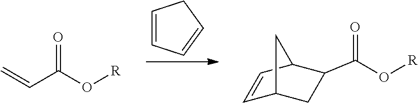

Freshly distilled cyclopentadiene (>1.3 eq) was added in drops to the particular ice-cold acrylate (1.0 eq). After 2 hours, the ice cooling was removed, and the mixture was stirred at room temperature. Since the reaction was exothermic, it was cooled with ice for another 15 minutes. Thereafter, no further exotherm was observed and the mixture was stirred at 40° C. overnight. Complete conversion was confirmed by means of ¹H-NMR spectroscopy. Excess cyclopentadiene and the by-product dicyclopentadiene were removed by flash column chromatography with Cy/EtOAc 1:0 (v/v), then 5:1 (v/v) as eluent. The end/exo ratio was determined by means of ¹H-NMR spectroscopy.

### Production of Mon9 (2-hydroxyethyl-endo,exo-5-norbornenecarboxylate)

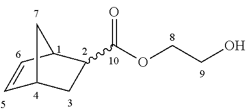 [SMILES: O=C(OCCO)C1CC2C=CC1C2]

Starting materials: Cyclopentadiene (45.0 mL, 35.40 g, 0.535 mol, 1.3 eq), 2-hydroxyethyl acrylate (40.0 mL, 44.33 g, 0.382 mol, 1.0 eq).

Yield: 66.78 g (96%), yellowish liquid, endo/exo: 80/20

Thin-layer chromatography (TLC): Rₜ=0.62 (Cy/EtOAc, 1:1, (v:v), detection: KMnO₄)

¹H-NMR (300 Hz, CDCl₃): δ=6.21-6.18 (m, 1H, 6_{endo}), 6.16-6.08 (m, 2H, 5ₑₓₒ, 6ₑₓₒ), 5.95-5.92 (m, 1H, 5_{endo}), 4.24-4.21 (t, 2H, 8ₑₓₒ), 4.17-4.14 (t, 2H, 8_{endo}), 3.84-3.77 (m, 2H, 9_{endo/exo}), 3.22 (s, 1H, 1_{endo}), 3.05 (s, 1H, 1ₑₓₒ), 3.02-2.96 (m, 1H, 2_{endo}), 2.91 (s, 1H, 4_{endo/exo}), 2.29-2.24 (m, 1H, 2ₑₓₒ), 2.08 (s, b, OH), 1.96-1.87 (m, 1H, 3a_{endo/exo}), 1.54-1.51 (d, 1H, 3ₑₓₒ), 1.45-1.35 (m, 2H, 7_{endo/exo}), 1.29-1.26 (d, 1H, 3b_{endo}) ppm.

¹³C-NMR (75 Hz, CDCl₃): δ=176.7 (1C, C_{q}, 10ₑₓₒ), 175.2 (1C, C_{q}, 10_{endo}), 138.2 (1C, 6ₑₓₒ), 137.9 (1C, 6_{endo}), 135.7 (1C, 5bₑₓₒ), 132.2 (1C, 5b, 66.1 (1C, 8ₑₓₒ), 65.9 (1C, 8_{endo}), 61.4 (2C, 9_{endo/exo}), 49.7 (1C, 7_{endo}), 46.7 (1C, 7ₑₓₒ), 46.4 (1C, 1ₑₓₒ), 45.8 (1C, 1_{endo}), 43.3 (1C, 2_{endo}), 43.1 (1C, 2ₑₓₒ), 42.6 (1C, 4_{endo}), 41.7 (1C, 4ₑₓₒ), 30.3 (1C, 3ₑₓₒ), 29.3 (1C, 3_{endo}) ppm.

### Example 1.2: Esterification Reactions

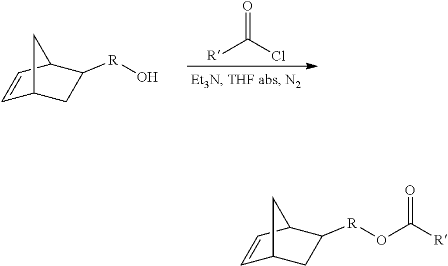

Hydroxy-functionalized norbornene (1.0 eq), absolute triethylamine (1.5 eq) and absolute tetrahydrofuran were mixed in an inert atmosphere with ice cooling. A pre-cooled, colorless acid chloride solution (1.5 eq: e.g. methacryloyl chloride) in THF abs, was added in drops over 45 minutes and the formation of a colorless precipitate (triethylammonium chloride) was observed. The reaction was continued for one hour in the ice bath, and then overnight at room temperature. Complete conversion to the relevant ester was monitored with TLC. The solvent was removed in vacuo and the residue was taken up in diethyl ether and an aqueous HCl solution (5 vol. %). The phases were separated and the organic phase was washed again with the HCl solution. The organic phase was then washed with an aqueous solution of sodium hydroxide (0.5 M) to remove residual free acid which had formed from the excess acid chloride during the work-up. The residual acid content was determined by ¹H-NMR spectroscopy. The solution was dried via sodium sulfate anhydride and the solvent was removed in vacuo.

### Production of Mon13 (2-(methacryloyloxy)ethyl-endo,exo-5-norbornenecarboxylate)

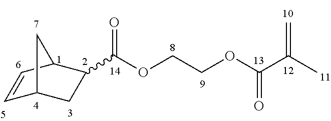 [SMILES: C=C(C)C(=O)OCCOC(=O)C1CC2C=CC1C2]

Starting materials: Mon9 (97.50 g, 0.535 mol, 1.0 eq), methacryloyl chloride (78.4 ml, 83.90 g, 0.803 mol, 1.5 eq), triethylamine (111.25 mL, 81.22 g, 0.803 mol, 1.5 eq), tetrahydrofuran (approx. 500 mL).

Yield: 124.56 g (93%), clear, yellowish liquid, endo/exo: 80/20

Thin-layer chromatography (TLC): R_{f}=0.71/0.79 (Cy/EtOAc, 3:1, (v:v), detection: KMnO₄)

¹H-NMR (300 Hz, CDCl₃): δ=6.22-6.15 (m, 1H, 6_{endo}), 6.15-6.06 (m, 1H, 5ₑₓₒ, 6ₑₓₒ; s, 1H, 10ₜᵣₐₙₛ), 5.94-5.86 (m, 1H, 5_{endo}), 5.59 (s, 1H, 10_{cis}), 4.40-4.17 (m, 4H, 8_{endo/exo}, 9_{endo/exo}), 3.19 (s, 1H, 1_{endo}), 3.03 (s, 1H, 1ₑₓₒ), 3.00-2.93 (m, 1H, 2_{endo}), 2.90 (s, 1H, 4_{endo/exo}) 2.28-2.21 (m, 1H, 2ₑₓₒ), 1.95 (s, 3H, 11), 1.92-1.83 (m, 1H, 3a_{endo}), 1.55-1.46 (d, 1H, 3aₑₓₒ), 1.46-1.31 (m, 2H, _{7endo/exo}, 1H, 3bₑₓₒ), 1.31-1.18 (d, 1H, 3b_{endo}) ppm.

¹³C-NMR (75 Hz, CDCl₃): δ=176.1 (1C, C_{q}, 14ₑₓₒ), 174.6 (1C, C_{q}, 10_{endo}), 167.2 (1C, C_{q}, 13), 138.2 (1C, 6ₑₓₒ), 138.0 (1C, 6_{endo}), 136.1 (1C, C_{q}, 12), 135.8 (1C, 5ₑₓₒ), 132.4 (1C, 5_{endo}), 126.10 (1C, 10), 62.6 (1C, 8_{endo}), 62.2 (1C, 8ₑₓₒ), 62.0 (1C, 9_{endo,exo}), 49.7 (1C, 7_{endo}), 46.8 (1C, 7ₑₓₒ), 46.5 (1C, 1ₑₓₒ), 45.8 (1C, 1_{endo}), 43.4 (1C, 2_{endo}), 43.2 (1C, 2ₑₓₒ), 42.7 (1C, 4_{endo}), 41.8 (1C, 4ₑₓₒ), 30.4 (1C, 3ₑₓₒ), 29.4 (1C, 3_{endo}), 18.4 (1C, 11) ppm.

### Production of Mon14 (endo,exo-5-norbornene-2-methylmethacrylate)

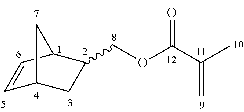

Starting materials: endo,exo-5-norbornene-2-methanol (97.37 mL, 100.0 g, 0.805 mol, 1.0 eq), methacryloyl chloride (118.0 ml, 126.26 g, 1.208 mol, 1.5 eq), triethylamine (167.44 mL, 122.23 g, 1.208 mol, 1.5 eq), tetrahydrofuran (approx. 500 mL).

Yield: 121.42 g (78%), clear, yellowish liquid, endo/exo: 76/24

Thin-layer chromatography (TLC): R_{f}=0.76 (Cy/EtOAc, 5:1, (v:v), detection: KMnO₄)

¹H-NMR (300 Hz, CDCl₃): δ=6.22-6.13 (m, 1H, 5_{endo}), 6.13-6.02 (m, 1H, 5ₑₓₒ, 6ₑₓₒ; s, 1H, 10ₜᵣₐₙₛ), 6.03-5.87 (m, 1H, 5_{endo}), 5.54 (s, 1H, 10_{cis}), 4.23-3.99 (m, 2H, 8ₑₓₒ), 3.99-3.64 (m, 2H, 8_{endo}), 2.90 (s, 1H, 1_{endo}), 2.82 (S, 1H, _{4endo/exo}), 2.72 (s, 1H, 1ₑₓₒ), 2.53-2.36 (m, 1H, 2_{endo}), 1.95 (s, 3H, 11), 1.91-1.80 (m, 1H, 3a_{endo}), 1.80-1.70 (m, 1H, 2ₑₓₒ), 1.51-1.41 (m, 1H, 7a_{endo}), 1.41-1.31 (m, 1H, 7a,bₑₓₒ), 1.31-1.24 (m, 1H, 7b_{endo}), 1.24-1.11 (m, 1H, 3a,bₑₓₒ), 0.65-0.52 (m, 1H, 3b_{endo}) ppm.

¹³C-NMR (75 Hz, CDCl₃): δ=167.7 (1C, C_{q}, 12ₑₓₒ), 167.6 (1C, C_{q}, 12_{endo}), 137.7 (1C, 5_{endo}), 137.1 (1C, 5ₑₓₒ), 136.7 (1C, C_{q}, 11_{endo}), 136.6 (1C, C_{q}, 11ₑₓₒ), 136.4 (1C, 6ₑₓₒ), 132.3 (1C, 6_{endo}), 125.4 (1C, 10ₑₓₒ), 125.2 (1C, 10_{endo}), 68.9 (1C, 8ₑₓₒ), 68.2 (1C, 8_{endo}), 49.5 (1C, 7_{endo}), 45.1 (1C, 7ₑₓₒ), 44.1 (1C, 1_{endo}), 43.8 (1C, 1ₑₓₒ), 42.3 (1C, 4_{endo}), 41.7 (1C, 4ₑₓₒ), 38.1 (1C, 2ₑₓₒ), 38.0 (1C, 2_{endo}), 29.7 (1C, 3ₑₓₒ), 29.1 (1C, 3_{endo}), 18.5 (1C, 9) ppm.

### Example 2: Single Cure RadP and Dual Cure RadP-ROMP—Shrinkage Tests

### Example 2.1: Production of the Formulations

The formulations used for the shrinkage tests were produced as follows:

2.1.1: Single Cure RadP Polymerization

An ultrasonic bath was used to dissolve BPO in the amount of monomer indicated in Table 1 in a 2.5 mL glass jar to form component A. The remaining amount of monomer was mixed with DMpT in another 2.5 mL glass jar to form component B. Component A was added to component B with a glass pipette, and the components were mixed homogeneously by shaking by hand. The amounts used of the constituents of components A and B are summarized in Table 1.

TABLE 1

| | Constituent | Amount in g |
|---|---|---|
| Component A | Monomer | 1.00 |
| | BPO | 0.015 |
| Component B | Monomer | 0.6 |
| | DMpT | 0.0075 |

2.1.2: Dual Cure RadP-ROMP Polymerization

An ultrasonic bath was used to dissolve BPO in the amount of monomer indicated in Table 2 in a 2.5 mL glass jar to form component A. The remaining amount of monomer was mixed with DMpT in another 2.5 mL glass jar to form component B.

An M20 stock solution was produced in dichloromethane (50 ppm, based on the total amount of monomer/100 μL). Component A and an aliquot of the M20 stock solution (component C) were added to component B at the same time, and the components were mixed homogeneously by shaking by hand.

The amounts used of the constituents of components A. B and C are summarized in Table 2.

TABLE 2

| | Constituent | Amount in g |
|---|---|---|
| Component A | Monomer | 1.00 |
| | BPO | 0.016 |
| Component B | Monomer | 0.5 |
| | DMpT | 0.0075 |
| Component C | M20 | 60 ppm relative |
| | | to the monomer |
| | | amount |
| | DCM | 100 μL |

### Example 2.2: Shrinkage Tests

Exactly 1.0 mL of the formulation was transferred to a 3.0 mL glass tube with a syringe. The glass tube was marked with a line at 1.0 mL and 2.0 mL filling levels. It was cured for 24 hours at room temperature. To determine the volumetric shrinkage, as much water as necessary was added to fill up to the 2.0 mL mark. This water was then measured with a graduated syringe.

Table 3 shows the monomers used and the results achieved therewith. Dual cure RadP-ROMP resulted in less shrinkage than single cure RadP with the reference monomers HEMA and BDDMA.

TABLE 3

| | Single cure RadP | | Dual cure RadP-ROMP | |
|---|---|---|---|---|
| | Shrinkage | Number of | Shrinkage | Number of |
| Monomer(s) | in % | samples | in % | samples |
| HEMA | 10.0 ± 0.0 | 5 | — | — |
| BDDMA | 4.2 ± 0.8 | 5 | — | — |
| HEMA + Mon9, | 3.8 ± 1.0 | 4 | 3.0 ± 1.6 | 5 |
| 1:1 molar ratio | | | | |
| HEMA + Mon13 | 6.0 ± 1.0 | 3 | 2.2 ± 0.8 | 5 |
| 1:1 molar ratio | | | | |
| HEMA + Mon14 | 8.5 ± 1.9 | 6 | 2.0 | 1 |
| 1:1 molar ratio | | | | |

### Example 3: Dual Cure RadP-ROMP Curing Tests to Determine the Influence of the Amount of Tempol and ROMP Initiator

Mon13 and Mon14 were mixed with Perkadox 20S in a 40 mL glass jar and stirred by hand in order to obtain a homogeneous mixture, component A. A stock solution of Tempol in BDDMA was produced by mixing these two compounds in a 40 mL glass jar with a magnetic stirrer.

The mixture was stirred at 50° C. for 15 minutes. The exact amount of M20 required was dissolved in an aliquot of the Tempol/BDDMA stock solution in a 20 mL glass jar. DMpT was added volumetrically to this solution, yielding component B. This component was taken up with a syringe and quickly added to component A. The two components were mixed homogeneously by shaking by hand. The weight proportions are listed in Table 4; the gelation process is noted in Table 5. The change in viscosity and the temperature development were observed.

TABLE 4

| | | | | Proportion based | |
|---|---|---|---|---|---|
| | | | | on the amount of | |
| | Constituent | Amount | | Mon13/Mon14 | |
| Component A | | | | | |
| | Mon13/Mon14 | 10 | g | 100 | wt. % |
| | Perkadox 20S | 3 | g | 30 | wt. % |
| Component B | | | | | |
| | M20 | see Table 5 | | 0-200 | ppm |
| | BDDMA | 0.5 | mL | 5 | wt. % |
| | Tempol | 23-35 | mg | 0.23-0.35 | wt. % |
| | DMpT | 111 | μL | 1.05 | wt. % |

TABLE 5

| | M20 | | Time from | |
|---|---|---|---|---|
| Tempol | [ppm | | mixing | Curing |
| [mg] | (mg)] | | [min] | behavior |
| Mon13 = monomer | | | | |
| 23 | 0 | (0) | 4 | Gelled |
| | | | 5.5 | Firmer, hot |
| 23 | 25 | (0.93) | 3.5 | Gelled |
| | | | 4 | Firm |
| 23 | 50 | (1.96) | 3.5 | Gelled |
| | | | 4 | Firm |
| 23 | 75 | (2.79) | 3.5 | Gelled |
| | | | 4 | Firm |
| | | | 4.5 | Hot |
| 23 | 100 | (3.72) | 3.5 | Gelled |
| | | | 4 | Firm |
| 23 | 150 | (5.58) | 2.5 | Gelled, 25° C. |
| | | | 3 | Firm, 40° C. |
| | | | 3.8 | Firm, hot |
| 23 | 200 | (7.44) | 2.5 | Gelled |
| | | | 3 | Firm, hot |
| 30 | 0 | (0) | 6.5 | Gelled |
| | | | 8 | Firm, hot |
| 35 | 0 | (0) | 9.3 | Gelled |
| | | | 12 | Firm, very elastic |
| Mon 14 = monomer | | | | |
| 23 | 0 | (0) | 16 | Gelled |
| | | | 20 | Firm |
| 23 | 50 | (2.42) | 1 | Increased viscosity |
| | | | 4 | High viscosity |
| | | | 9 | Gelled |
| | | | 10.5 | Firm, hot |
| 23 | 60 | (2.91) | 0.5 | Increased viscosity |
| | | | 2 | High viscosity |
| | | | 7 | Gelled |
| | | | 10.5 | Firm, 40° C. |
| | | | 11 | Hot |
| 23 | 75 | (3.63) | 2 | High viscosity |
| | | | 7 | Gelled |
| | | | 8.5 | Firm, hot |
| 23 | 100 | (4.84) | 0.5 | Increased viscosity |
| | | | 3 | High viscosity |
| | | | 7 | Firm, hot |

These results show that the curing behavior can be adjusted by varying the amount of ROMP initiator and inhibitor (Tempol).

### Example 4: Dual Cure RadP-ROMP Versus Single Cure RadP—Gel Timer Measurements

Gel timer measurements were carried out to determine the gel time of various formulations with (“filled”) and without fillers (“unfilled”).

4.1: Production of the Formulations

The unfilled and filled formulations (Table 6) were prepared in the same way as in Example 3. The total amount of fillers was added to component A to produce the filled formulations. The ingredients were mixed homogeneously in a speed mixer and component A was stored at a constant 25° C. Then component B was added and homogenized again with a speed mixer. The mixing ratio was A:B:C 20.20:60 (w/w). The same procedure was used to produce the unfilled formulations, but the fillers were omitted.

TABLE 6

| | | | Proportion based | |
|---|---|---|---|---|
| | | Amount | on the amount of | |
| | Constituent | in g | Mon13/Mon14 | |
| Component A | Mon13/Mon14 | 50.00 | 100 | wt. % |
| | Perkadox 208 | 16.0 | 30 | wt. % |
| Component B | M20 | 0.0093/0.0121 | 50 | ppm |
| | BDDMA | 1.023 | 2 | wt. % |
| | Tempol | 0.175/0.116 | 0.35/0.23 | wt. % |
| | DMpT | 0.522 | 1.05 | wt. % |
| Fillers | Cab-O-Sil TS-720 | 3.90 | 7.8 | wt. % |
| | Quartz sand F32 | 48.35 | 96.7 | wt. % |
| | Millisil W12 | 26.65 | 53.3 | wt. % |

4.2: Gel Timer Measurements

The GELNORM®-Geltimer PS-1 from H. Saur Laborbedarf (“gel timer”) was used. The formulations were poured into glass tubes, which were placed in a thermostat at a constant 25° C. (Eco RE2025 from Lauda Dr. R. Wobser GmbH & Co. KG). The temperature profile was measured using the PT 100 sensor from Pico Technology. In addition, measurements were taken in parallel in another glass tube with a mechanical sensor (MK-B-10).

For this purpose, the mixture was poured into a test tube after the addition of the initiator, up to a height of 4 cm below the rim, the test tube being kept at a temperature of 25° C. (DIN 16945, DIN EN ISO 9396). A glass rod or spindle was moved up and down at 10 strokes per minute in the mixture. When measuring the temperature profile, the gel time corresponded to the period of time following the addition of the initiator after which a temperature of 35° C. (i.e. +10′C based on the starting temperature 25° C.) or 50° C. was measured in the mixture. During the mechanical measurement, the gel time was determined as the period of time following the addition of the initiator after which the sensor could no longer be moved.

The results are shown in Table 7 (GTT: gel time from temperature profile; GTM: gel time with mechanical sensor), the chemical anchor HIT-HY150 (component A contains 1,4-butanediol dimethacrylate, 2-hydroxypropyl methacrylate and a proprietary urethane dimethacrylate; component B contains dibenzoyl peroxide) from Hilti was used as a reference:

TABLE 7

| | | unfilled/ | GTT_{+10° C.} | GTT_{50° C.} | GTM |
|---|---|---|---|---|---|
| System | Monomer | filled | [min] | [min] | [min] |
| Dual cure | Mon13 | unfilled | 5.83 | 6.65 | 6.35 |
| RadP-ROMP | | filled | 9.57 | 10.68 | — |
| | Mon14 | unfilled | 3.73 | 7.21 | 7.15 |
| | | filled | 11.37 | 12.15 | — |
| RadP | HIT-HY-150 | unfilled | 5.10 | 5.55 | 4.31 |
| (reference) | | filled | 5.12 | 5.35 | 5.20 |

“unfilled”: without fillers;
“filled”: with fillers

The gel time of the dual cure samples was mostly a little longer than the gel time of the radically initiated reference. However, it was within the time frame required for its planned use as a chemical anchor.

### Example 5: Dual Cure RadP-ROMP and Single Cure RadP—Pull-Out Tests and Gel Timer Measurements when Used in 10+1 Cartridges

Filled dual cure RadP-ROMP formulations (Table 8) were tested. A single cure formulation with Mon13 as the monomer, in which the M20 was missing, served as a comparison in the pull-out tests. The pull-out force, the shear strength and the gel time were tested.

5.1: Production of the Samples

The composition of the components is shown in Table 8:

TABLE 8

| | | Amount based | | Proportion based | |
|---|---|---|---|---|---|
| | | on the entire | | on the amount of | |
| | Constituent | component | | Mon13 | |
| Component A | Mon13 | 31 | wt. % | 100 | wt. % |
| | Perkadox 20S | 9 | wt. % | 30 | wt. % |
| | Cab-O-Sil TS-720 | 3 | wt. % | | |
| | Quartz sand F32 | 37 | wt. % | | |
| | Millisil W12 | 20 | wt. % | | |
| Total comp. A | | 100 | wt. % | | |
| | | approx. 300 mL | | | |
| Component B | M20 [only with | 0.1 | wt. % | 50 | ppm |
| | dual cure] | | | | |
| | BDDMA | 36 | wt. % | 11.7 | wt. % |
| | Tempol | 0.7 | wt. % | 0.23 | wt. % |
| | DMPT | 3.2 | wt. % | 1.05 | wt. % |
| | Cab-O-Sil TS-720 | 3 | wt. % | | |
| | Quartz sand F32 | 37 | wt. % | | |
| | Millisil W12 | 20 | wt. % | | |
| Total comp. B | | 100 | wt. % | | |

approx. 30 mL

The relevant monomer was mixed with Perkadox 20S in a speed mixer for 30 s. The fillers were added and the speed mixer was used to make component A homogeneous. Component B was produced analogously to the procedure in Example 3, with SpeedMixer boxes instead of glass jars. The finished components were then poured into the relevant chamber of 2-component (“2C”) 10+1 cartridges (component A in the 10-chamber, component B in the 1-chamber). These were stored at room temperature, except for the gel timer measurement, for which they were stored at a constant 25° C. for at least one hour. The formulations were mixed and applied using a squeeze gun with a static mixer tip.

5.2: Pull-Out Tests

Boreholes (diameter: 14 mm) in concrete (C20/25) were filled with the squeeze gun and a metal threaded anchor rod (diameter: 12 mm) was then inserted to a depth of 72 mm (embedding depth). The pull-out tests were carried out after a curing time of 48 h, using a pull-out device F-100-4 from Kindsmüller with a Hoko 100 B cylinder.

Anchor rods having an M8 thread having the following geometry were used to carry out the measurements:
Undercut depth: 0.35+/−0.02 mm
Undercut width: 2 mm

The shear strength determined from these measurements is defined as the quotient of the maximum force upon failure and the shear surface of the anchor rod used (approx. 2765 mm²).

It was tested in dry (“F1ref”) and wet (“F1b”) concrete; three boreholes were filled and tested for each tested composition. The epoxy-amine system HIT-RE 500 V1 and the radically polymerized methacrylate system HIT-HY 200-A from Hilti were used as reference systems.

The results for pull-out force and shear strength are shown in Table 9.

TABLE 9

| | | Pull-out | Shear |
|---|---|---|---|
| | Borehole | force | strength |
| System | condition | [kN] | [N/mm²] |
| RE 500 V1 | F1ref | | 33.0 ± 1.5 |
| (Epoxy amine) | F1b | | 28.0 ± 1.4 |
| HY 200-A | F1ref | 90.5 ± 1.0 | 34.1 ± 0.5 |
| (RadP) | F1b | 65.9 ± 6.9 | 24.5 ± 2.2 |
| Single cure RadP | F1ref | 38.2 ± 4.0 | 14.1 ± 1.5 |
| Mon 13 | F1b | 25.3 ± 2.1 | 9.0 ± 0.9 |
| Dual cure RadP | F1ref | 41.7 ± 4.5 | 15.3 ± 1.7 |
| Mon 13 | F1b | 46.5 ± 3.6 | 17.1 ± 1.3 |

The result shows that dual cure RadP-ROMP does not achieve the pull-out forces and shear strength of the reference systems, however, the values are sufficient for fastening in boreholes, in addition, the dual cure system exhibits a significant advantage that is likely to be related to the low-shrinkage ROMP: the equivalence (or even improvement) of the values of F1b compared to F1ref. This means that this system is very robust with respect to wet boreholes. This is not the case with the other formulations, where F1b is always significantly smaller than F1ref.

5.3: Gel Timer Measurement

The gel timer measurement was carried out as described in Example 4.2. The chemical anchor HIT-HY150 from Hilti again served as a reference. The results are shown in Table 10:

TABLE 10

| | unfilled/ | Tempol | GTT_{+10° C.} | GTT_{50° C.} | GTM |
|---|---|---|---|---|---|
| System | filled | [wt. %] | [min] | [min] | [min] |
| RadP-ROMP | unfilled | 0.35 | 5.83 | 6.65 | 6.35 |
| Mon13 | filled | | 4.78 | 5.53 | 5.00 |
| | | | 4.97 | 5.65 | 5.00 |
| HY150 | unfilled | 0.46 | 5.10 | 5.55 | 4.31 |
| | filled | | 5.12 | 5.35 | 5.20 |

These results show that the gel time values are comparable to those of the reference.

### Example 6: Stability Tests with the ROMP Catalyst and the ROMP Monomer EsterMon

6.1: ROMP with M20 (Table 11, Line 1):

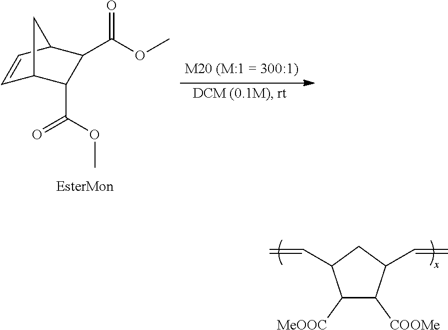

400 mg (1.9 mmol, 1 eq) EsterMon was dissolved in 18 mL dichloromethane. A stock solution of initiator (5.9 mg/mL, 50 ppm) in dichloromethane was produced. The polymerization reactions were started either immediately or 15 hours after the stock solutions had been produced. The reaction mixture was stirred at room temperature and the progress was monitored by TLC (R_{f}(EsterMon)=0.62 (Cy/EtOAc, 3:1, (v:v), detection: KMnO₄)). After complete conversion, the reaction was quenched with 400 μL ethyl vinyl ether and stirred for a further 20 min. The solution was concentrated in vacuo. The residual solution was added in drops to rapidly stirred, cooled methanol. The precipitated, colorless polymer was dried in vacuo. If the reaction was still incomplete after 24 hours, the monomer:polymer ratio was determined with ¹H-NMR before quenching and work-up.

6.2: ROMP of the Compound EsterMon in the Presence of the Individual Components of the RadP Radical Starter System (Table 11, Lines 2 to 4):

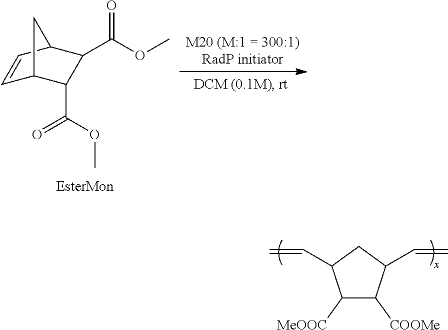

These polymerizations were carried out analogously to the ROMP of EsterMon described above (Table 47, entry 1). Two procedures were used to add the individual components:
Mixing EsterMon with the individual test substances in DCM, adding the M20 stock solution to start the polymerization (“0 h”);
Adding the individual test substances to a freshly prepared M20 stock solution, storage for 15 h. followed by the addition of the relevant M20 stock solution to an Ester-Mon solution in DCM (“15 h”).

TABLE 11

| | Amount | | | Time until | Ratio of |
|---|---|---|---|---|---|
| | (based on | | | complete | monomer:polymer |
| | the monomer | Storage | | conversion of | after |
| Reagent | amount) | time | | the monomer | 24 h |
| M20 | 5.9 mg | 0 | h | 1 h or 0.66 h | — |
| | (50 ppm) | 15 | h | >24 h | 1:300 |
| | | | | | (almost complete |
| | | | | | conversion) |
| +BPO | 4.0 mg | 0 | h | 0.66 | — |
| | (1.0 wt. %) | 15 | h | >24 h | 1:0 |
| | | | | | (no conversion) |
| +DMpT | 2.77 μL | 0 | h | 0.66 | — |
| | (0.5 wt. %) | 15 | h | 1.33 | |
| +Tempol | 0.32 mg | 0 | h | 0.66 | — |
| | (0.08 wt. %) | 15 | h | >24 h | 1:0.06 |
| | | | | | (minimal |
| | | | | | conversion) |

It was found that M20 is unstable when stored with BPO or Tempol (see the 15 h storage time values in lines 2 (“M20+BPO”) and 4 (“M20+Tempol”)). The ROMP initiator must therefore be stored separately from the radical initiator (here: BPO) and from Tempol and should also be separate from these components in a 2C system.

It was also shown that DMpT can stabilize the M20. After 15 h of storage in the presence of DMpT, the process was significantly faster than after 15 h of storage without the presence of DMpT (compare the 15 h values in lines 1 (“M20”) and 3 (“M20+DMpT”)). The storage stability of the M20 can thus be increased by adding DMpT or a comparable reagent.

1: A two-component system for producing a polymer, comprising:
a component A comprising
a monomer which contains a strained cycloolefin as a structural element (ROMP monomer), and
a radical initiator, and
a component B comprising
a methacrylate,
a catalyst for ring-opening metathesis polymerization (ROMP initiator), and
a radical accelerator.

2: The two-component system according to claim 1, wherein the strained cycloolefin is a monocyclic or bicyclic C₃-C₁₆ cycloolefin having one or two double bonds.

3: The two-component system according to claim 2, wherein the strained cycloolefin is selected from the group consisting of norbornene, norbornadiene, tricyclo[5.2.1.0^{2,6}]deca-3,8-diene (dicyclopentadiene), tetracyclo[6.2.1.1.0]-dodecene, and their oxa derivatives, in which a C atom in the strained cycloolefin structural element is replaced by O.

4: The two-component system according to claim 3, wherein the strained cycloolefin is a norbornene.

5: The two-component system according to claim 1, wherein the ROMP initiator is a Grubbs catalyst.

6: The two-component system according to claim 5, wherein the ROMP initiator is selected from the group consisting of: 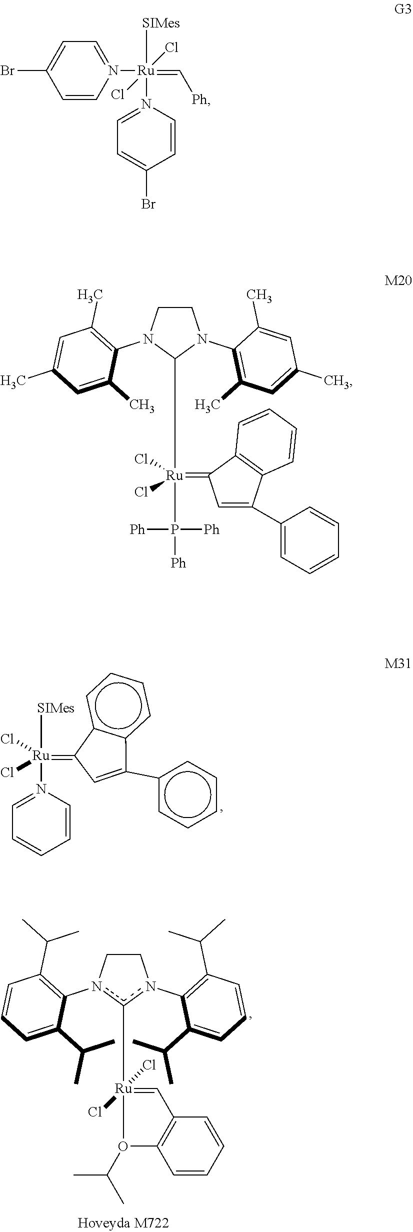
and combinations of two or more thereof.

7: The two-component system according to claim 1, wherein the radical initiator is a peroxide.

8: The two-component system according to claim 1, wherein the methacrylate in the component B is selected from the group consisting of methyl methacrylate, 2-hydroxyethyl methacrylate (HEMA), and 1,4-butanediol dimethacrylate (BDDMA).

9: The two-component system according to claim 1, wherein the radical accelerator is an aromatic amine.

10: The two-component system according to claim 1, wherein the component B additionally comprises an inhibitor.

11: The two-component system according to claim 1, wherein at least one of the components A or B contains at least one filler, and wherein the at least one filler can be in the form of sand, powder, or shaped bodies.

12: The two-component system according to claim 11, wherein the at least one filler is a fumed silica after-treated in a non-polar manner.

13: A method, comprising:
fastening an anchor in a borehole with the two-component system according to claim 1.

14: The method according to claim 13, wherein the anchor is made of steel or iron, and
wherein the borehole is a borehole in a mineral or metal substrate.

15: A method for the chemical fastening of an anchor in a borehole, the method comprising:
mixing components of the two-component composition as defined in claim 1, in the borehole to fasten the anchor.

16: The two-component system according claim 7, wherein the peroxide is benzoyl peroxide.

17: The two component system according to claim 9, wherein the radical accelerator is selected from the group consisting of: N,N-di-iso-propanol-p-toluidine (DiPpT), N,N-dimethyl-p-toluidine (DMpT), and N,N-diethyl-p-toluidine (DEpT).

18: The two-component system according to claim 10, wherein the inhibitor is 4-hydroxy-2,2,6,6-tetramethylpiperidinyloxyl (Tempol).

19: The two-component system according to claim 11, wherein the at least one filler is selected from the group consisting of quartz, glass, corundum, porcelain, earthenware, light spar, barite, gypsum, talc, chalk, and a mixture thereof.

20: The two-component system according to claim 14, wherein the mineral or metal substrate is selected from the group consisting of concrete, aerated concrete, brickwork, limestone, sandstone, natural stone, glass, and steel.
